SEQUENCE LISTING (98 sequences)

SEQ ID NO: 1 - DNA, 24 bp, Artificial sequence
  Artificially synthesized oligonucleotide primer
gagtatttaggaggtataagaatt

SEQ ID NO: 2 - DNA, 23 bp, Artificial sequence
  Artificially synthesized oligonucleotide primer
atcaaaaactaacataaacccct

SEQ ID NO: 3 - DNA, 25 bp, Artificial sequence
  Artificially synthesized oligonucleotide primer
gtaaggagattatgtttatttttgg

SEQ ID NO: 4 - DNA, 21 bp, Artificial sequence
  Artificially synthesized oligonucleotide primer
cctcattaatcccataactat

SEQ ID NO: 5 - DNA, 26 bp, Artificial sequence
  Artificially synthesized oligonucleotide primer
ttagtggggtatttttatttgtatgg

SEQ ID NO: 6 - DNA, 26 bp, Artificial sequence
  Artificially synthesized oligonucleotide primer
aaatatcctaaaaatacaaactacac

SEQ ID NO: 7 - DNA, 26 bp, Artificial sequence
  Artificially synthesized oligonucleotide primer
gtgtggtatttttatgtatagttagg

SEQ ID NO: 8 - DNA, 24 bp, Artificial sequence
  Artificially synthesized oligonucleotide primer
gtttatagaagtaggggtggtttt

SEQ ID NO: 9 - DNA, 20 bp, Artificial sequence
  Artificially synthesized oligonucleotide primer
aatcccccacacctaaattc

SEQ ID NO: 10 - DNA, 20 bp, Artificial sequence
  Artificially synthesized oligonucleotide primer
taaggtgagtggtttaggat

SEQ ID NO: 11 - DNA, 28 bp, Artificial sequence
  Artificially synthesized oligonucleotide primer
gagagtatgtaaagttagagttgtgttg

SEQ ID NO: 12 - DNA, 26 bp, Artificial sequence
  Artificially synthesized oligonucleotide primer
ataatacaacaacaacaataacaatc

SEQ ID NO: 13 - DNA, 29 bp, Artificial sequence
  Artificially synthesized oligonucleotide primer
taaagatagtttagatatggaattttggg

SEQ ID NO: 14 - DNA, 29 bp, Artificial sequence
  Artificially synthesized oligonucleotide primer
gtgttaaggtatattatgttagtgttagg

SEQ ID NO: 15 - DNA, 27 bp, Artificial sequence
  Artificially synthesized oligonucleotide primer
tacaacccttccctcactccaaaaatt

SEQ ID NO: 16 - DNA, 28 bp, Artificial sequence
  Artificially synthesized oligonucleotide primer
atattatgttagtgttaggaaggattgt

SEQ ID NO: 17 - DNA, 26 bp, Artificial sequence
  Artificially synthesized oligonucleotide primer
gatttgggatataaaaggttaatgag

SEQ ID NO: 18 - DNA, 26 bp, Artificial sequence
  Artificially synthesized oligonucleotide primer
tcattaaaaacacaaacctcctttac

SEQ ID NO: 19 - DNA, 26 bp, Artificial sequence
  Artificially synthesized oligonucleotide primer
ttttagattttgagggttttaggttg

SEQ ID NO: 20 - DNA, 26 bp, Artificial sequence
  Artificially synthesized oligonucleotide primer
aatcccttaaaaatcatctttcacac

SEQ ID NO: 21 - DNA, 29 bp, Artificial sequence
  Artificially synthesized oligonucleotide primer
tatgtaatatgatatagtttagaaattag

SEQ ID NO: 22 - DNA, 29 bp, Artificial sequence
  Artificially synthesized oligonucleotide primer
aataaacccaaatctaaaatattttaatc

SEQ ID NO: 23 - DNA, 28 bp, Artificial sequence
  Artificially synthesized oligonucleotide primer
aatttgtgtgatgtttgtaattatttgg

SEQ ID NO: 24 - DNA, 28 bp, Artificial sequence
  Artificially synthesized oligonucleotide primer
ataaaatacactttcactactaaaatcc

SEQ ID NO: 25 - DNA, 24 bp, Artificial sequence
  Artificially synthesized oligonucleotide primer
tgggttgaaatattgggtttattt

SEQ ID NO: 26 - DNA, 23 bp, Artificial sequence
  Artificially synthesized oligonucleotide primer
ctaaaaccaaatatccaaccata

SEQ ID NO: 27 - DNA, 25 bp, Artificial sequence
  Artificially synthesized oligonucleotide primer
ccaccctctaaccttaacctctaac

SEQ ID NO: 28 - DNA, 24 bp, Artificial sequence
  Artificially synthesized oligonucleotide primer
gaggatgttttttaagtttttttt

SEQ ID NO: 29 - DNA, 25 bp, Artificial sequence
  Artificially synthesized oligonucleotide primer
cccacactcatatcaatataataac

SEQ ID NO: 30 - DNA, 25 bp, Artificial sequence
  Artificially synthesized oligonucleotide primer
aatgtttatggtggattttgtaggt

SEQ ID NO: 31 - DNA, 20 bp, Artificial sequence
  Artificially synthesized oligonucleotide primer
atccgagcaactggtttgtg

SEQ ID NO: 32 - DNA, 20 bp, Artificial sequence
  Artificially synthesized oligonucleotide primer
caatcccaccctctagcctt

SEQ ID NO: 33 - DNA, 21 bp, Artificial sequence
  Artificially synthesized oligonucleotide primer
ggtgcaatggctgtcttgtcc

SEQ ID NO: 34 - DNA, 21 bp, Artificial sequence
  Artificially synthesized oligonucleotide primer
tcacaaaccagttgctcggat

SEQ ID NO: 35 - DNA, 28 bp, Artificial sequence
  Artificially synthesized oligonucleotide primer
tctttagatcagaggatgccccctaagc

SEQ ID NO: 36 - DNA, 27 bp, Artificial sequence
  Artificially synthesized oligonucleotide primer
aagcctcctaccctacccaccccctat

SEQ ID NO: 37 - DNA, 17 bp, Artificial sequence
  Artificially synthesized oligonucleotide primer
ggccggtgagtgagcga

SEQ ID NO: 38 - DNA, 21 bp, Artificial sequence
  Artificially synthesized oligonucleotide primer
cgggttttataggacgccaca

SEQ ID NO: 39 - DNA, 20 bp, Artificial sequence
  Artificially synthesized oligonucleotide primer
atccgagcaactggtttgtg

SEQ ID NO: 40 - DNA, 20 bp, Artificial sequence
  Artificially synthesized oligonucleotide primer
gggacgtctggacaggacaa

SEQ ID NO: 41 - DNA, 20 bp, Artificial sequence
  Artificially synthesized oligonucleotide primer
aggatgccccctaagctttc

SEQ ID NO: 42 - DNA, 20 bp, Artificial sequence
  Artificially synthesized oligonucleotide primer
gggtccaccatggacattgt

SEQ ID NO: 43 - DNA, 19 bp, Artificial sequence
  Artificially synthesized oligonucleotide primer
acatcgccaatcagcttgg

SEQ ID NO: 44 - DNA, 23 bp, Artificial sequence
  Artificially synthesized oligonucleotide primer
agaaccatactcgaaccacatcc

SEQ ID NO: 45 - DNA, 18 bp, Artificial sequence
  Artificially synthesized oligonucleotide primer
ctgggaacgcctcatcaa

SEQ ID NO: 46 - DNA, 20 bp, Artificial sequence
  Artificially synthesized oligonucleotide primer
catcttctgcttcctggcaa

SEQ ID NO: 47 - DNA, 22 bp, Artificial sequence
  Artificially synthesized oligonucleotide primer
ttttcagaaatcccttccctcg

SEQ ID NO: 48 - DNA, 21 bp, Artificial sequence
  Artificially synthesized oligonucleotide primer
cgttcccagaattcgatgctt

SEQ ID NO: 49 - DNA, 22 bp, Artificial sequence
  Artificially synthesized oligonucleotide primer
tgctccaaggtgaagctgaaag

SEQ ID NO: 50 - DNA, 24 bp, Artificial sequence
  Artificially synthesized oligonucleotide primer
gtagggcatgttgaacactttatg

SEQ ID NO: 51 - DNA, 21 bp, Artificial sequence
  Artificially synthesized oligonucleotide primer
gcagagttggccatgaagatg

SEQ ID NO: 52 - DNA, 21 bp, Artificial sequence
  Artificially synthesized oligonucleotide primer
tcagtttgtctgttcggaccg

SEQ ID NO: 53 - DNA, 19 bp, Artificial sequence
  Artificially synthesized oligonucleotide primer
ttggaagaacttgcccacg

SEQ ID NO: 54 - DNA, 21 bp, Artificial sequence
  Artificially synthesized oligonucleotide primer
ggctgcgatcgatatgcatct

SEQ ID NO: 55 - DNA, 21 bp, Artificial sequence
  Artificially synthesized oligonucleotide primer
ggcctatctttgcaactccca

SEQ ID NO: 56 - DNA, 19 bp, Artificial sequence
  Artificially synthesized oligonucleotide primer
ggtgctggacggggaaact

SEQ ID NO: 57 - DNA, 21 bp, Artificial sequence
  Artificially synthesized oligonucleotide primer
acgagcgccaggtacctactc

SEQ ID NO: 58 - DNA, 22 bp, Artificial sequence
  Artificially synthesized oligonucleotide primer
tgtctattgtgcgccacctatg

SEQ ID NO: 59 - DNA, 22 bp, Artificial sequence
  Artificially synthesized oligonucleotide primer
ggaacctcacaaacgcctgtaa

SEQ ID NO: 60 - DNA, 20 bp, Artificial sequence
  Artificially synthesized oligonucleotide primer
atgcgaacgacttcttcgcc

SEQ ID NO: 61 - DNA, 19 bp, Artificial sequence
  Artificially synthesized oligonucleotide primer
acgtttggagagggacacc

SEQ ID NO: 62 - DNA, 21 bp, Artificial sequence
  Artificially synthesized oligonucleotide primer
ctttccgctgtaacctttctg

SEQ ID NO: 63 - DNA, 19 bp, Artificial sequence
  Artificially synthesized oligonucleotide primer
ttgcctgaggatggctgtg

SEQ ID NO: 64 - DNA, 21 bp, Artificial sequence
  Artificially synthesized oligonucleotide primer
gcccaaaccttagtcctccat

SEQ ID NO: 65 - DNA, 20 bp, Artificial sequence
  Artificially synthesized oligonucleotide primer
ggaaagcactcctccccatt

SEQ ID NO: 66 - DNA, 21 bp, Artificial sequence
  Artificially synthesized oligonucleotide primer
gccaacacaggcttttcctct

SEQ ID NO: 67 - DNA, 21 bp, Artificial sequence
  Artificially synthesized oligonucleotide primer
ggagcacattcagcacacgat

SEQ ID NO: 68 - DNA, 24 bp, Artificial sequence
  Artificially synthesized oligonucleotide primer
gtcgaatggaggtatctttcctga

SEQ ID NO: 69 - DNA, 21 bp, Artificial sequence
  Artificially synthesized oligonucleotide primer
gcagcgttttcctgtacagct

SEQ ID NO: 70 - DNA, 24 bp, Artificial sequence
  Artificially synthesized oligonucleotide primer
gtgtccatccccattcattttatc

SEQ ID NO: 71 - DNA, 20 bp, Artificial sequence
  Artificially synthesized oligonucleotide primer
cataacgaggctgagctcgg

SEQ ID NO: 72 - DNA, 21 bp, Artificial sequence
  Artificially synthesized oligonucleotide primer
cctgtatgttgggcaggtcac

SEQ ID NO: 73 - DNA, 20 bp, Artificial sequence
  Artificially synthesized oligonucleotide primer
ctgcaaggacatgagcccac

SEQ ID NO: 74 - DNA, 20 bp, Artificial sequence
  Artificially synthesized oligonucleotide primer
gaggcagtccctgcaatgac

SEQ ID NO: 75 - DNA, 20 bp, Artificial sequence
  Artificially synthesized oligonucleotide primer
catggccaccacattctcaa

SEQ ID NO: 76 - DNA, 21 bp, Artificial sequence
  Artificially synthesized oligonucleotide primer
gcggccagtaccctcataaag

SEQ ID NO: 77 - DNA, 21 bp, Artificial sequence
  Artificially synthesized oligonucleotide primer
ctctggagaaagccagggttc

SEQ ID NO: 78 - DNA, 19 bp, Artificial sequence
  Artificially synthesized oligonucleotide primer
cactccagcatgggcttca

SEQ ID NO: 79 - DNA, 22 bp, Artificial sequence
  Artificially synthesized oligonucleotide primer
gaggagagacgtggagaaatgg

SEQ ID NO: 80 - DNA, 19 bp, Artificial sequence
  Artificially synthesized oligonucleotide primer
ggatccggtggaactggaa

SEQ ID NO: 81 - DNA, 19 bp, Artificial sequence
  Artificially synthesized oligonucleotide primer
gctgaagagcaagcatgcg

SEQ ID NO: 82 - DNA, 23 bp, Artificial sequence
  Artificially synthesized oligonucleotide primer
tcttcaccaggaggtcaactttc

SEQ ID NO: 83 - DNA, 23 bp, Artificial sequence
  Artificially synthesized oligonucleotide primer
gaccagcctgacagatttctatc

SEQ ID NO: 84 - DNA, 20 bp, Artificial sequence
  Artificially synthesized oligonucleotide primer
ctcctgacccacagcagaag

SEQ ID NO: 85 - DNA, 21 bp, Artificial sequence
  Artificially synthesized oligonucleotide primer
tgcgctgcaggttatgaaact

SEQ ID NO: 86 - DNA, 21 bp, Artificial sequence
  Artificially synthesized oligonucleotide primer
ctgctctggcagcatcatgaa

SEQ ID NO: 87 - DNA, 22 bp, Artificial sequence
  Artificially synthesized oligonucleotide primer
cgagcacctgaaattcttctgg

SEQ ID NO: 88 - DNA, 19 bp, Artificial sequence
  Artificially synthesized oligonucleotide primer
cgggtcaccatttcagcaa

SEQ ID NO: 89 - DNA, 22 bp, Artificial sequence
  Artificially synthesized oligonucleotide primer
tgcagtctacatacgcaacacc

SEQ ID NO: 90 - DNA, 19 bp, Artificial sequence
  Artificially synthesized oligonucleotide primer
gaggcttccgacggaacat

SEQ ID NO: 91 - DNA, 22 bp, Artificial sequence
  Artificially synthesized oligonucleotide primer
cagaaagcctctttttcgtgga

SEQ ID NO: 92 - DNA, 18 bp, Artificial sequence
  Artificially synthesized oligonucleotide primer
ggaatgaaaagcctgccg

SEQ ID NO: 93 - DNA, 22 bp, Artificial sequence
  Artificially synthesized oligonucleotide primer
catacgcctgcagagttaagca

SEQ ID NO: 94 - DNA, 24 bp, Artificial sequence
  Artificially synthesized oligonucleotide primer
gatcacatgtctcgatcccagtag

SEQ ID NO: 95 - DNA, 22 bp, Artificial sequence
  Artificially synthesized oligonucleotide primer
cgacgatgctccccgggctgta

SEQ ID NO: 96 - DNA, 30 bp, Artificial Sequence
  Artificially synthesized oligonucleotide primer
tctctttgatgtcacgcacgatttccctct

SEQ ID NO: 97 - DNA, 19 bp, Artificial Sequence
  Artificially synthesized oligonucleotide primer
acatcgccaatcagcttgg

SEQ ID NO: 98 - DNA, 23 bp, Artificial Sequence
  Artificially synthesized oligonucleotide primer
agaaccatactcgaaccacatcc

(19) United States
(12) Patent Application Publication
(10) Pub. No.: US 2012/0045758 A1
(43) Pub. Date: Feb. 23, 2012

(54) IMPRINTING IN VERY SMALL EMBRYONIC-LIKE (VSEL) STEM CELLS

(76) Inventors: Magdalena Kucia, Louisville, KY (US); Dong-Myung Shin, Louisville, KY (US); Mariusz Ratajczak, Louisville, KY (US); Janina Ratajczak, Louisville, KY (US)

(21) Appl. No.: 13/129,352

(22) PCT Filed: Nov. 16, 2009
(86) PCT No.: PCT/US2009/064612

§ 371 Date: Aug. 26, 2011

Related U.S. Application Data
(60) Provisional application No. 61/199,345, filed on Nov. 14, 2008.

(51) Int. Cl. C12Q 1/68

(52) U.S. Cl. 435/6.11; 435/6.12

(57) ABSTRACT

Methods for determining a degree of pluripotency in a first putative stem cell relative to a second putative stem cell are provided. In some embodiments the methods include comparing the imprinting status in the first versus the second putative stem cell of a locus selected from among Igf2-H19, Rasgrf1, lgf2R, Kcnq1, and Peg1/Mest. Also provided are methods for distinguishing very small embryonic like (VSEL) stem cells from hematopoietic stem cells (HSCs) and mesenchymal stem cells (MSCs), methods for isolating VSELs from sources expected to include VSELs, methods for assessing the purity of a very small embryonic like stem cell (VSEL) preparation, and kits that include oligonucleotide primers that can be employed in the practice of the claimed methods.

### CROSS REFERENCE TO RELATED APPLICATION

The presently disclosed subject matter claims the benefit of U.S. Provisional Patent Application Ser. No. 61/199,345, filed Nov. 14, 2008; the disclosure of which is incorporated herein by reference in its entirety.

### GRANT STATEMENT

This work was supported by grants P20 RR018733, R01 CA106281-01, and R01 DK074720 from the National Institutes of Health of the United States of America. Accordingly, the United States Government has certain rights in the presently disclosed subject matter.

### TECHNICAL FIELD

The presently disclosed subject matter relates in some embodiments to methods for determining relative pluripotencies among different types of stem cells. More particularly, the presently disclosed subject matter relates in some embodiments to comparing imprinting status of a locus selected from the group comprising Igf2-H19, Rasgrf1,Igf2R, Kcnq1, and Peg1/Mest, wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus are indicative of a more pluripotent state.

### BACKGROUND

The use of pluripotent cells and derivatives thereof has gained increased interest in medical research, particularly in the area of providing reagents for treating tissue damage either as a result of genetic defects, injuries, and/or disease processes. Ideally, pluripotent cells that are capable of differentiating into the affected cell types could be transplanted into a subject in need thereof, where they would interact with the organ microenvironment and supply the necessary cell types to repair the injury.

Considerable effort has been expended to isolate pluripotent cells from a number of different tissues for use in regenerative medicine. For example, U.S. Pat. No. 5,750,397 to Tsukamoto at al. discloses the isolation and growth of human hematopoietic stem cells that are reported to be capable of differentiating into lymphoid, erythroid, and myelomonocytic lineages. U.S. Pat. No. 5,736,396 to Bruder et al. discloses methods for lineage-directed differentiation of isolated human mesenchymal stem cells under the influence of appropriate growth and/or differentiation factors. The derived cells can then be introduced into a host for mesenchymal tissue regeneration or repair.

One area of intense interest relates to the use of embryonic stem (ES) cells, which have been shown in mice to have the potential to differentiate into all the different cell types of the animal. Mouse ES cells are derived from cells of the inner cell mass of early mouse embryos at the blastocyst stage, and other pluripotent and/or totipotent cells have been isolated from germinal tissue (e.g., primordial germ cells; PGCs). The ability of these pluripotent and/or totipotent stem cells to proliferate in vitro in an undifferentiated state, retain a normal karyotype, and retain the potential to differentiate to derivatives of all three embryonic germ layers (endoderm, mesoderm, and ectoderm) makes these cells attractive as potential sources of cells for use in regenerative therapies in post-natal subjects. The development of human ES (hES) cells has not been as successful as the advances that have been made with mouse ES cells, however, and ethical concerns have been raised with respect to the method by which hES cells are generated.

Additionally, it would be beneficial to be able to isolate and purify stem cells and/or other pluripotent cells from a subject that could thereafter be further purified and/or manipulated in vitro before being reintroduced into the subject for treatment purposes. The use of a subject's own cells would also have advantages, particularly with respect to obviating the need to employ adjunct immunosuppressive therapy, thereby maintaining the competency of the subject's immune system. Alternatively or in addition, it would be beneficial to be able to confirm that appropriate cells have been isolated, and/or assess the purity of stem cells and/or other pluripotent cells in a cell population isolated from a subject.

As such, the search for pluripotent cell types from adult animals is an ongoing effort, which has seen some degree of progress. For example, mesenchymal stem cells (MSCs) are one such cell type. MSCs have been shown to have the potential to differentiate into several lineages including bone (Haynesworth et al. (1992) 13 Bone 81-88), cartilage (Mackay et al. (1998) 4 Tissue Eng 415-28; Yoo et al. (1998) 80 J Bone Joint Surg Am 1745-57), adipose tissue (Pittenger et al. (2000) 251 Curr Top Microbiol Immunol 3-11), tendon (Young et al. (1998) 16 J Orthop Res 406-13), muscle, and stroma (Caplan et al. (2001) 7 Trends Mol Med 259-64).

Another population of cells, multipotent adult progenitor cells (MAPCs), has also been purified from bone marrow (BM; Reyes et al. (2001) 98 Blood 2615-2625; Reyes & Verfaillie (2001) 938 Ann NY Acad Sci 231-235). These cells have been shown to be capable of expansion in vitro for more than 100 population doublings without telomere shortening or the development of karyotypic abnormalities. MAPCs have also been shown to be able to differentiate under defined culture conditions into various mesenchymal cell types (e.g., osteoblasts, chondroblasts, adipocytes, and skeletal myoblasts), endothelium, neuroectoderm cells, and more recently, into hepatocytes (Schwartz et al. (2000) 109 J Clin Invest 1291-1302).

Additionally, hematopoietic stem cells (HSCs) have been reported to be able to differentiate into numerous cell types. BM hematopoietic stem cells have been reported to be able to “transdifferentiate” into cells that express early heart (Orlic et al. (2003) 7 Pediatr Transplant 86-88; Makino et al. (1999) 103 J Clin Invest 697-705), skeletal muscle (Labarge & Blau (2002) 111 Cell 589-601; Corti et al. (2002) 277 Exp Cell Res 74-85), neural (Sanchez-Ramos (2002) 69 Neurosci Res 880-893), liver (Petersen et al. (1999) 284 Science 1168-1170), or pancreatic cell (Ianus et al. (2003) 111 J Clin Invest 843-850; Lee & Stoffel (2003) 111 J Clin Invest 799-801) markers. In vivo experiments in humans also demonstrated that transplantation of CD34 peripheral blood (PB) stem cells led to the appearance of donor-derived hepatocytes (Korbling et al. (2002) 346 N Engl J Med 738-746), epithelial cells (Korbling et al. (2002) 346 N Engl J Med 738-746), and neurons (Hao et al. (2003) 12 J Hematother Stem Cell Res 23-32). Additionally, human BM-derived cells have been shown to contribute to the regeneration of infarcted myocardium (Stamm et al. (2003) 361 Lancet 45-46).

These reports have been interpreted as evidence for the existence of the phenomenon of transdifferentiation or plasticity of adult stem cells. However, the concept of transdifferentiation of adult tissue-specific stem cells is currently a topic of extensive disagreement within the scientific and medical communities (see e.g., Lemischka (2002) 30 Exp Hematol 848-852; Holden & Vogel (2002) 296 Science 2126-2129). Studies attempting to reproduce results suggesting transdifferentiation with neural stem cells have been unsuccessful (Castro et al. (2002) 297 Science 1299). It has also been shown that the hematopoietic stem/progenitor cells (HSPC) found in muscle tissue originate in the BM (McKinney-Freeman et al. (2002) 99 Proc Natl Acad Sci USA 1341-1346; Geiger et al. 100 Blood 721-723; Kawada & Ogawa (2001) 98 Blood 2008-2013). Additionally, studies with chimeric animals involving the transplantation of single HSPCs into lethally irradiated mice demonstrated that transdifferentiation and/or plasticity of circulating HPSC and/or their progeny, if it occurs at all, is an extremely rare event (Wagers et al. (2002) 297 Science 2256-2259).

Thus, there continues to be a need for new approaches to generate, identify, and/or confirm the purity of populations of transplantable cells suitable for a variety of applications, including but not limited to treating injury and/or disease of various organs and/or tissues.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

The presently disclosed subject matter provides methods for determining a degree of pluripotency in a first putative stem cell relative to a second putative stem cell. In some embodiments, the methods comprise comparing imprinting statuses of one or more loci selected from the group consisting of Igf2-H19, Rasgrf1, Igf2R, Kcnq1, and Peg1/Mest between the first putative stem cell and the second putative stem cell, wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus are indicative of a more pluripotent state. In some embodiments, the first and second putative stem cells are selected from the group consisting of very small embryonic like stem cells (VSELs), hematopoietic stem cells (HSCs), and mesenchymal stem cells (MSCs).

The presently disclosed subject matter also provides methods for distinguishing a very small embryonic like stem cell (VSEL) from a hematopoietic stem cell (HSC) or a mesenchymal stem cell (MSC). In some embodiments, the methods comprise comparing an imprinting status of one or more loci of the VSEL selected from the group consisting of Igf2-H19, Rasgrf1, Igf2R, Kcnq1, and Peg1/Mest to the same one or more loci in an HSC or an MSC, wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus relative to levels of methylation at these loci in an HSC or an MSC are indicative of VSELs.

The presently disclosed subject matter also provides methods for isolating a very small embryonic like stem cell (VSEL) from a source expected to comprise VSELs. In some embodiments, the methods comprise (a) isolating a plurality of CD45^{neg}/lin^{neg} in cells that are Sca-1⁺ or CD34⁺ from the source; and (b) isolating a subset of cells from the plurality of CD45^{neg}/lin^{neg} cells that are Sca-1⁺ or CD34⁺, wherein the subset of cells are characterized by one or more of hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus as compared to the fraction of cells remaining in the plurality of CD45^{neg}/lin^{neg} cells that are Sca-1⁺ or CD34⁺. In some embodiments, the methods further comprise fractionating the cells to identify cells that are Oct-4⁺, CXCR4⁺, and/or SSEA-1⁺.

The presently disclosed subject matter also provides methods for assessing the purity of a very small embryonic like stem cell (VSEL) preparation. In some embodiments, the methods comprise (a) providing a first preparation suspected of comprising VSELs; and (b) comparing an imprinting profile of cells of the first preparation with respect to one or more loci selected from the group consisting of Igf2-H19, Rasgrf1,Igf2R, Kcnq1, and Peg1/Mest to an imprinting profile of a second preparation of VSELs with respect to the same one or more loci, wherein relative to the second preparation, hypermethylation at the Igf2-H19 locus, hypermethylation at the Rasgrf1 locus, hypomethylation at the Igf2R locus, hypomethylation at the Kcnq1 locus, and hypomethylation at the Peg1/Mest locus relative to levels of methylation at these loci in the second preparation is indicative of the first preparation being less pure with respect to VSELs than the second preparation. In some embodiments, the presently disclosed methods further comprise isolating the first preparation from a source that comprises VSELs and at least one other stem cell type selected from the group consisting of hematopoietic stem cells (HSCs) and mesenchymal stem cells (MSCs).

In some embodiments of any of the disclosed methods, the hypomethylation at the Rasgrf1 locus comprises hypomethylation at a differentially methylated region (DMR) of the Rasgrf1 promoter, the hypermethylation at the Igf2R locus comprises hypomethylation at a DMR2 region of the IgfR2 promoter, the hypermethylation at the Kcnq1 locus comprises hypermethylation of a KvDMR region of the Kcnq1 promoter, and/or the hypermethylation at the Peg1/Mest locus comprises hypermethylation of a DMR region of the Peg1/Mest promoter.

The presently disclosed subject matter also provides compositions for use in the presently disclosed methods. In some embodiments, the compositions comprise a kit comprising a plurality of oligonucleotide primers, wherein the oligonucleotide primers specifically bind to a subsequence of a differentially methylated region (DMR) in a nucleic acid or bind to a nucleotide sequence that flanks a DMR in a nucleic acid, wherein the oligonucleotide primers can be used to assay the methylation status of at least one methylated nucleotide present within the DMR. In some embodiments, the DMR is a human DMR. In some embodiments, the DMR is present in a locus selected from the group consisting of an Igf2-H19 locus, a Rasgrf1 locus, an Igf2R locus, a Kcnq1 locus, and a Peg1/Mest locus. In some embodiments, the plurality of oligonucleotide primers are designed to assay the DMR using a technique selected from the group consisting of bisulfite sequencing, carrier chromatin-immunoprecipitation (ChIP), and quantitative ChIP (qChIP). In some embodiments, at least one of the plurality of oligonucleotides primers comprises a nucleotide sequence of any of SEQ ID NOs: 1-96.

Thus, it is an object of the presently disclosed subject matter to provide methods for determining a degree of pluripotency in a first putative stem cell relative to a second putative stem cell.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1G depict the results of experiments showing that the Oct4 promoter in VSELs is transcriptionally active.

FIG. 1A depicts a strategy of fluorescence-activated cell sorting (FACS) for isolation of VSELs (Lin^{neg}/Sca-1⁺/CD45^{neg}) and HSCs (Lin^{neg}/Sca-1⁺/CD45⁺) from murine bone marrow (BM). As shown in the Figure, the lymphocyte gate (R1) was extended to the left to include small sized stem cells (SCs).

FIG. 1B is a photograph of an agarose gel of RT-PCR products showing expression of Oct4 mRNA in VSELs, HSCs, STs, and ESC-D3 cells. β-Actin was included as a loading control. The control reactions were performed without RTase (lanes indicated as “-”).

FIG. 1C depicts the results of immunostaining VSELs for Oct4 and SSEA-1 protein. Oct4 was localized to the nucleus as shown by nuclear staining with DAPI.

FIG. 1D is a schematic depicting the location of CpG sites (open-circles) in the Oct4 promoter and the locations of primers Oct4-S1 and Oct4-S2 employed for ChIP assays.

FIG. 1E depicts the results of bisulfite sequencing of DNA methylation of the Oct4 promoter in VSELs, HSCs, STs, and EBs. Methylated and unmethylated CpG sites are shown in filled circles and open circles, respectively. The number under each bisulfite sequencing profile indicates the percentage of CpG sites that were methylated.

FIG. 1F is a series of photographs of agarose gels showing the results of ChIP analyses of the Oct4 promoter in VSELs, HSCs, MNCs, and ES cells (ESC) mixed with THP-1 cells. The top panel depicts regular ChIP analyses, in which the amplification of the β-actin promoter was performed as a control reaction of the endogenous housekeeping gene. For FIG. 1F, the PCR reactions were conducted in bound (B) and unbound (UB) fractions using two different primer sets for the Oct4 promoters (Oct4-S1, Oct4-S2) specific to mouse sequences. In the bottom panels, ChIP analysis of H3Ac (left panel) and H3K9me2 (right panel) are depicted. For these panels, the corresponding PCR reactions were conducted with the indicated number of cycles (Cs). THP-1 cells were also tested alone as a negative control, and no PCR products were seen in the corresponding lanes. Rabbit IgG (IgG) antibody was used as negative immunoprecipitation control. D.W.: distilled water.

FIG. 1G is a pair of bar graphs showing the results of quantitative ChIP analyses for the Oct4 promoter to evaluate its association with H3Ac and H3K9me2 histones in VSELs, HSCs, MNCs, and ESC. In the quantitative ChIP assay, the enrichment of each histone modification was calculated as the ratio of the value from the bound fraction (B) to that from the unbound fraction (UB). Fold differences are shown as the mean±S.D. from at least four independent experiments. ** p<0.01 compared to BM-MNC.

FIGS. 2A-2D depict the results of analyses of the epigenetic status of the Nanog promoter in VSELs, HSCs, STs, and ESC-D3 cells.

FIG. 2A is a photograph of an agarose gel showing the results of RT-PCR analysis of Nanog mRNA in VSELs, HSCs, STs, and ESC-D3 cells. β-actin was used as a loading control, and assays performed in the absence of RTase (−) were used as the negative control.

FIG. 2B depicts the results of bisulfite sequencing of DNA methylation of the Nanog promoter. Methylated and unmethylated CpG sites are shown in filled and open circles, respectively. The numbers under each bisulfite sequencing result indicates the percentage of methylated CpG sites.

FIGS. 2C and 2D depict the results of regular (FIG. 2C) and quantitative (FIG. 2D) ChIP analyses of H3Ac and H3K9me2 modifications in the Nanog promoter. In regular ChIP analysis (FIG. 2C), the PCR was run for the indicated number of cycles (C) using ChIP products from bound (B) and unbound (UB) fraction. In quantitative ChIP analysis (FIG. 2D), the enrichment of each histone modification was calculated as the ratio of the value from B to that from the UB fraction and the fold differences are shown as the mean±S.D. from at least four independent experiments. ** p<0.01 compared to BMMNC.

FIGS. 3A-3E depict the results of analysis showing the erasure of genomic imprinting for paternally-methylated imprinted genes in VSELs.

FIG. 3A is a schematic diagram of DMRs present within the Igf2-H19, Rasgrf1, and Meg3 loci. The upper and bottom arrows represent the maternally and paternally initiated transcription sites, respectively, for the indicated genes. E: Enhancer.

FIGS. 3B-3D depict the bisulfite sequencing profiles of DNA methylation of DMRs for the Igf2-H19 (FIG. 3B), Rasgrf1 (FIG. 3C), and Meg3 (FIG. 3D) loci. The percentage of methylated CpG sites was shown by employing bisulfite modification and sequencing results. Unlike DMRs of Igf2-H19 and Rasgrf1, there was little difference in DNA methylation for intergenic (IG)-DMR for Meg3 locus.

FIG. 3E depicts the results of COBRA assay analysis of the Igf2-H19 DMR1 by Taql restriction enzyme (upper panel) and IG-DMR for Meg3 locus by BstUl restriction enzyme (lower panel). The unmethylated DNA (UMe) was not cleaved in contrast to methylated DNA (Me), indicating a sequence change in the corresponding site recognized by a restriction enzyme after bisulfite reaction.

FIGS. 4A-4F depict the results of assays showing the hypermethylated status of DMRs of VSELs in maternally-methylated imprinted genes.

FIG. 4A is a schematic diagram of DMRs for the Kcnq1 and Igf2R loci. DMRs for the Kcnq1 and Igf2R loci are located in promoter for antisense-transcripts, Lit1 and Air, respectively.

FIGS. 4B, 4C, 4E, and 4F depict bisulfite-sequencing results of DNA methylation patterns of DMRs for the Kcnq1 (FIG. 4B), Igf2R (FIG. 4C), Peg1 (FIG. 4E), and SNRPN (FIG. 4F) loci. The percentage of methylated CpG sites is shown under each of the bisulfite-sequencing results.

FIG. 4D is a photograph of an agarose gel showing the results of COBRA assay of Igf2R DMR2 cleaved by Taql restriction enzyme (upper panel) and KvDMR cleaved by BstUl restriction enzyme (lower panel). The unmethylated DNA (UMe) was not cleaved in contrast to methylated DNA (Me).

FIGS. 5A-5G are a series of bar graphs and a photograph showing that the unique genomic imprinting patterns in VSELs affect the expression level of imprinted-genes.

FIGS. 5A-5D and 5F are bar graphs showing the results of RQ-PCR analysis of Igf2-H19 (FIG. 5A) and Rasgrf1 (FIG. 5B), which DMRs were hypomethylated in VSELs, and the maternally-methylated imprinted genes, Igf2R (FIG. 5C), p57^{KIP2} (FIG. 5D), and Peg1 (FIG. 5F). Of note, VSELs express little of the antisense transcripts Air (FIG. 5C) and Lit1 (FIG. 5D) for the Igf2R and Kcnq1 loci, respectively. The relative expression levels are represented as the fold-difference to the value determined in STs, and are shown as the mean±S.D. from at least three independent experiments on different samples of double-sorted VSELs, HSCs, STs, and ESC-D3. *p<0.05, **p<0.01 compared to ST.

FIG. 5E is a pair of photographs showing immunostaining of in VSELs. The p57^{KIP2} protein was localized in the nucleus.

FIG. 5G is a series of bar graphs showing the results of assaying for the expression of various CDKIs and Cdks in VSELs, HSCs, STs, and ESC-D3s. RQ-PCR analysis of various CDKIs (p21^{Cip1}, p18^{INK4c}, p57^{KIP2},a) and Cdks (Cdk2, Cdk4, and Cdk6) is depicted. The relative expression levels are represented as fold-differences with respect to expression in STs (y-axes), and are shown as mean±S.D. from at least four independent experiments performed on different cell populations. *p<0.05, ** p<0.01 compared to ST.

FIGS. 6A and 6B are a series of bar graphs and a series of photographs, respectively, showing that VSELs express a high level of Dnmts.

FIG. 6A is a series of bar graphs showing the results of RQ-PCR analyses of Dnmt1, 3b, 3a, and related protein Dnmt3L. The relative expression levels, are represented as the fold-difference to the value of STs and shown as the mean±S.D. from at least three independent experiments performed on double-sorted VSELs, HSCs, STs, and ESC-D3 cells. *p<0.05, **p<0.01 compared to ST.

FIG. 6B is a series of photographs showing the results of immunostaining VSELs for Dnmt1 and Dnmt3b proteins. DAPI staining was included to visualize nuclei, and the images merged with DAPI (merged) are shown in the right half of each panel. Both Dnmts were localized to the nuclei.

FIGS. 7A-7F depict the strategy and results of experiments designed to assay recovery from repressive genomic imprinting during VSEL-DS formation.

FIG. 7A is a schematic that depicts the experimental strategy outlines in more detail in EXAMPLE 4 hereinbelow. Briefly, VSELs were freshly isolated from the BM of GFP transgenic mice (GFP-Tg) and used to grow VSEL-DSs. GFP⁺ cells were sorted from the cultures.

FIG. 7B is a plot showing a summary of bisulfite-sequencing results (see FIGS. 7C-7E) of DNA methylation in the imprinted-genes DMRs and the Oct4 promoter in freshly isolated VSELs and VSEL-DSs (at 5, 7, 11 days). The paternally-imprinted DMRs (H19, Rasgrf1) were marked as blue lines and the maternally-imprinted DMRs (Igf2R, KvDMR, Peg1) were marked as red lines. The dashed red line indicates the normal methylation status (50%).

FIGS. 7C-7E depict the DNA methylation statuses of DMRs during VSEL-DS formation. Bisulfite-sequencing profiles of DNA methylation in the promoter of Oct4 (FIG. 7C), the paternally-methylated DMRs H19 (FIG. 7D, top panel), Rasgrf1 (FIG. 7D, bottom panel), and the maternally-methylated DMRs Igf2R, Kcnq1, and Peg1 (FIG. 7E) in freshly isolated VSELs and in VSEL-DS, formed at days 5, 7, and 11 of co-culture with C2C12 cells. GFP⁺ VSELs were plated and GFP⁺ cells from VSEL-DS were purified by FACS.

FIG. 7F is a schematic diagram of a proposed model for epigenetic reprogramming of VSELs deposited in adult tissues during development and their potential activation in response to tissue and organ injury.

### BRIEF DESCRIPTION OF THE SEQUENCE LISTING

SEQ ID NOs: 1-42 are the sequences of oligonucleotide primers that were employed in the bisulfite-sequencing, regular ChIP, and quantitative ChIP (qChIP) assays described in the EXAMPLES. The sequences of these oligonucleotide primers are set forth in Table 1.

TABLE 1
Sequences of the Oligonucleotide Primers
Employed in the Bisulfite-sequencing, Regular
ChIP, and Quantitative ChIP (qChIP) Assays

| Outer | | Inner | |
|---|---|---|---|
| Primers | Sequence | Primers | Sequence |
| H19- | GAGTATTTAGGAGGT | H19- | GTAAGGAGATTATGT |
| OF | ATAAGAATT | IF | TTATTTTTGG |
| | (SEQ ID NO: 1) | | (SEQ ID NO: 3) |
| H19- | ATCAAAAACTAACAT | H19- | CCTCATTAATCCCAT |
| OR | AAACCCCT | IR | AACTAT |
| | (SEQ ID NO: 2) | | (SEQ ID NO: 4) |
| Igf2R- | TTAGTGGGGTATTTT | Igf2R- | GTGTGGTATTTTTAT |
| OF | TATTTGTATGG | IF | GTATAGTTAGG |
| | (SEQ ID NO: 5) | | (SEQ ID NO: 7) |
| Igf2R- | AAATATCCTAAAAAT | Igf2R- | AAATATCCTAAAAAT |
| OR/IR | ACAAACTACAC | OR/IR | ACAAACTACAC |
| | (SEQ ID NO: 6) | | (SEQ ID NO: 6) |
| KvDMR- | GTTTATAGAAGTAGG | KvDMR- | TAAGGTGAGTGGTTT |
| OF | GGTGGTTTT | IF | AGGAT |
| | (SEQ ID NO: 8) | | (SEQ ID NO: 10) |
| KvDMR- | AATCCCCCACACCTA | KvDMR- | AATCCCCCACACCTA |
| OR/IR | TAATC | OR/IR | AATTC |
| | (SEQ ID NO: 9) | | (SEQ ID NO: 9) |
| Rasgrf1- | GAGAGTATGTAAAGT | Rasgrf1- | TAAAGATAGTTTAGA |
| OF | TAGAGTTGTGTTG | IF | TATGGAATTTTGGG |
| | (SEQ ID NO: 11) | | (SEQ ID NO: 13) |
| Rasgrf1- | ATAATACAACAACAA | Rasgrf1- | ATAATACAACAACAA |
| OR/IR | CAATAACAATC | OR/IR | CAATAACAATC |
| | (SEQ ID NO: 12) | | (SEQ ID NO: 12) |
| Meg3- | GTGTTAAGGTATATT | Meg3- | ATATTATGTTAGTGT |
| IG- | ATGTTAGTGTTAGG | IG- | TAGGAAGGATTGT |
| DMR- | (SEQ ID NO: 14) | DMR- | (SEQ ID NO: 16) |
| OF | | IF | |
| Meg3- | TACAACCCTTCCCTC | Meg3- | TACAACCCTTCCCTC |
| IG- | ACTCCAAAAATT | IG- | ACTCCAAAAATT |
| DMR- | (SEQ ID NO: 15) | DMR- | (SEQ ID NO: 15) |
| OR/IR | | OR/IR | |
| Peg 1- | GATTTGGGATATAAA | Peg1- | TTTTAGATTTTGAGG |
| OF | AGGTTAATGAG | IF | GTTTTAGGTTG |
| | (SEQ ID NO: 17) | | (SEQ ID NO: 19) |
| Peg 1- | TCATTAAAAACACAA | Peg 1- | AATCCCTTAAAAATC |
| OR | ACCTCCTTTAC | IR | ATCTTTCACAC |
| | (SEQ ID NO: 18) | | (SEQ ID NO: 20) |

| | | | |
|---|---|---|---|
| SNRPN- | TATGTAATATGATAT | SNNRP- | AATTTGTGTGATGTT |
| OF | AGTTTAGAAATTAG | IF | TGTAATTATTTGG |
| | (SEQ ID NO: 21) | | (SEQ ID NO: 23) |
| SNRPN- | AATAAACCCAAATCT | SNNRP- | ATAAAATACACTTTC |
| OR | AAAATATTTTAATC | IR | ACTACTAAAATCC |
| | (SEQ ID NO: 22) | | (SEQ ID NO: 24) |
| Oct4- | TGGGTTGAAATATTG | Oct4- | TGGGTTGAAATATTG |
| OF/IF | GGTTTATTT | OF/IF | GGTTTATTT |
| | (SEQ ID NO: 25) | | (SEQ ID NO: 25) |
| Oct4- | CTAAAACCAAATATC | Oct4- | CCACCCTCTAACCTT |
| OR | CAACCATA | IR | AACCTCTAAC |
| | (SEQ ID NO: 26) | | (SEQ ID NO: 27) |
| Nanog- | GAGGATGTTTTTTAA | Nanog- | AATGTTTATGGTGGA |
| OF | GTTTTTTTT | IF | TTTTGTAGGT |
| | (SEQ ID NO: 28) | | (SEQ ID NO: 30) |
| Nanog- | CCCACACTCATATCA | Nanog- | CCCACACTCATATCA |
| OR/IR | ATATAATAAC | OR/IR | ATATAATAAC |
| | (SEQ ID NO: 29) | | (SEQ ID NO: 29) |

ChIP or qChIP Primers

| | | | |
|---|---|---|---|
| Oct4- | ATCCGAGCAACTGGT | Oct4- | CAATCCCACCCTCTA |
| S1- | TTGTG | S1- | GCCTT |
| F | (SEQ ID NO: 31) | R | (SEQ ID NO: 32) |
| Oct4- | GGTGCAATGGCTGTC | Oct4- | TCACAAACCAGTTGC |
| S2- | TTGTCC | S2- | TCGGAT |
| F | (SEQ ID NO: 33) | R | (SEQ ID NO:34) |
| Nanog- | TCTTTAGATCAGAGG | Nanog- | AAGCCTCCTACCCTA |
| CHIP- | ATGCCCCCTAAGC | CHIP- | CCCACCCCCTAT |
| F | (SEQ ID NO: 35) | R | (SEQ ID NO: 36) |
| βActin- | GGCCGGTGAGTGAGC | βActin- | CGGGTTTTATAGGAC |
| CHIP- | GA | CHIP- | GCCACA |
| F | (SEQ ID NO: 37) | R | (SEQ ID NO: 38) |
| Oct4- | ATCCGAGCAACTGGT | Oct4- | GGGACGTCTGGACAG |
| qCHIP- | TTGTG | qCHIP- | GACAA |
| F | (SEQ ID NO: 39) | R | (SEQ ID NO: 40) |
| Nanog- | AGGATGCCCCCTAAG | Nanog- | GGGTCCACCATGGAC |
| qCHIP- | CTTTC | qCHIP- | ATTGT |
| F | (SEQ ID NO: 41) | R | (SEQ ID NO: 42) |

TABLE 2
Sequences of Oligonucleotide Primers Employed
for RT-PCR and qRT-PCR Analyses

| Forward | | Reverse | |
|---|---|---|---|
| Primers | Sequence | Primers | Sequence |
| Oct4- | ACATCGCCAATCAGC | Oct4- | AGAACCATACTCGAA |
| (q)RT-F | TTGG | (q)RT-R | CCACATCC |
| | (SEQ ID NO: 43) | | (SEQ ID NO: 44) |
| Nanog- | CTGGGAACGCCTCAT | Nanog- | CATCTTCTGCTTCCT |
| RT-F | CAA | RT-R | GGCAA |
| | (SEQ ID NO: 45) | | (SEQ ID NO: 46) |
| Nanog- | TTTTCAGAAATCCCT | Nanog- | CGTTCCCAGAATTCG |
| qRT-F | TCCCTCG | qRT-R | ATGCTT |
| | (SEQ ID NO: 47) | | (SEQ ID NO: 48) |
| H19- | TGCTCCAAGGTGAAG | H19- | GTAGGGCATGTTGAA |
| RT-F | CTGAAAG | RT-R | CACTTTATG |
| | (SEQ ID NO: 49) | | (SEQ ID NO: 50) |
| H19- | TGCTCCAAGGTGAAG | H19- | GCAGAGTTGGCCATG |
| qRT-F | CTGAAAG | qRT-R | AAGATG |
| | (SEQ ID NO: 49) | | (SEQ ID NO: 51) |
| Igf2- | TCAGTTTGTCTGTTC | Igf2- | TTGGAAGAACTTGCC |
| (q)RT-F | GGACCG | (q)RT-R | CACG |
| | (SEQ ID NO: 52) | | (SEQ ID NO: 53) |
| Igf2R- | GGCTGCGATCGATAT | Igf2R- | GGCCTATCTTTGCAA |
| (q)RT-F | GCATCT | (q)RT-R | CTCCCA |
| | (SEQ ID NO: 54) | | (SEQ ID NO: 55) |
| Air- | GGTGCTGGACGGGGA | Air- | ACGAGCGCCAGGTAC |
| RT-F | AACT | RT-R | CTACTC |
| | (SEQ ID NO: 56) | | (SEQ ID NO: 57) |
| Air- | TGTCTATTGTGCGCC | Air- | GGAACCTCACAAACG |
| qRT-F | ACCTATG | qRT-R | CCTGTAA |
| | (SEQ ID NO: 58) | | (SEQ ID NO: 59) |
| p57^{KIP2}- | ATGCGAACGACTTCT | p57^{KIP2}- | ACGTTTGGAGAGGGA |
| (q)RT-F | TCGCC | (q)RT-R | CACC |
| | (SEQ ID NO: 60) | | (SEQ ID NO: 61) |
| Lit1- | CTTTCCGCTGTAACC | Lit1- | TTGCCTGAGGATGGC |
| RT-F | TTTCTG | RT-R | TGTG |
| | (SEQ ID NO: 62) | | (SEQ ID NO: 63) |
| Lit1- | GCCCAAACCTTAGTC | Lit1- | GGAAAGCACTCCTCC |
| (q)RT-F | CTCCAT | (q)RT-R | CCATT |
| | (SEQ ID NO: 64) | | (SEQ ID NO: 65) |
| Rasgrf1- | GCCAACACAGGCTTT | RasgrfT- | GGAGCACATTCAGCA |
| (q)RT-F | TCCTCT | (q)RT-R | CACGAT |
| | (SEQ ID NO: 66) | | (SEQ ID NO: 67) |
| Peg1- | GTCGAATGGAGGTAT | Peg1- | GCAGCGTTTTCCTGT |
| RT-F | CTTTCCTGA | RT-R | ACAGCT |
| | (SEQ ID NO: 68) | | (SEQ ID NO: 69) |
| Peg1- | GTGTCCATCCCCATT | Peg1- | GCAGCGTTTTCCTGT |
| qRT-F | CATTTTATC | qRT-R | ACAGCT |
| | (SEQ ID NO: 70) | | (SEQ ID NO: 69) |
| Dnmt1- | CATAACGAGGCTGAG | Dnmt1- | CCTGTATGTTGGGCA |
| RT-F | CTCGG | RT-R | GGTCAC |
| | (SEQ ID NO: 71) | | (SEQ ID NO: 72) |
| Dnmt1- | CTGCAAGGACATGAG | Dnmt1- | CCTGTATGTTGGGCA |
| qRT-F | CCCAC | qRT-R | GGTCAC |
| | (SEQ ID NO: 73) | | (SEQ ID NO: 72) |
| Dnmt3a- | GAGGCAGTCCCTGCA | Dnmt3a- | CATGGCCACCACATT |
| RT-F | ATGAC | RT-R | CTCAA |
| | (SEQ ID NO: 74) | | (SEQ ID NO: 75) |
| Dnmt3a- | GAGGCAGTCCCTGCA | Dnmt3a- | GCGGCCAGTACCCTC |
| qRT-F | ATGAC | qRT-R | ATAAAG |
| | (SEQ ID NO: 74) | | (SEQ ID NO: 76) |
| Dnmt3b- | CTCTGGAGAAAGCCA | Dnmt3b- | CACTCCAGCATGGGC |
| (q)RT-F | GGGTTC | (q)RT-R | TTCA |
| | (SEQ ID NO: 77) | | (SEQ ID NO: 78) |
| Dnmt3L- | GAGGAGAGACGTGGA | Dnmt3L- | GGATCCGGTGGAACT |
| RT-F | GAAATGG | RT-R | GGAA |
| | (SEQ ID NO: 79) | | (SEQ ID NO: 80) |
| Dnmt3L- | GCTGAAGAGCAAGCA | Dnmt3L- | TCTTCACCAGGAGGT |
| (q)RT-F | TGCG | (q)RT-R | CAACTTTC |
| | (SEQ ID NO: 81) | | (SEQ ID NO: 82) |
| p21^{Cip1}- | GACCAGCCTGACAGA | p21^{Cip1}- | CTCCTGACCCACAGC |
| (q)RT-F | TTTCTATC | (q)RT-R | AGAAG |
| | (SEQ ID NO: 83) | | (SEQ ID NO: 84) |
| p18^{INK4C}- | TGCGCTGCAGGTTAT | p18^{INK4C}- | CTGCTCTGGCAGCAT |
| (q)RT-F | GAAACT | (q)RT-R | CATGAA |
| | (SEQ ID NO: 85) | | (SEQ ID NO: 86) |
| Cdk2- | CGAGCACCTGAAATT | Cdk2- | CGGGTCACCATTTCA |
| qRT-F | CTTCTGG | qRT-R | GCAA |
| | (SEQ ID NO: 87) | | (SEQ ID NO: 88) |
| Cdk4- | TGCAGTCTACATACG | Cdk4- | GAGGCTTCCGACGGA |
| qRT-F | CAACACC | qRT-R | ACAT |
| | (SEQ ID NO: 89) | | (SEQ ID NO: 90) |
| Cdk6- | CAGAAAGCCTCTTTT | Cdk6- | GGAATGAAAAGCCTG |
| qRT-F | TCGTGGA | qRT-R | CCG |
| | (SEQ ID NO: 91) | | (SEQ ID NO: 92) |
| β2-M¹- | CATACGCCTGCAGAG | β2-M- | GATCACATGTCTCGA |
| qRT-F | TTAAGCA | qRT-R | TCCCAGTAG |
| | (SEQ ID NO: 93) | | (SEQ ID NO: 94) |
| βActin- | CGACGATGCTCCCCG | βActin- | CTCTTTGATGTCACG |
| RT-F | GGCTGTA | RT-R | CACGATTTCCCTCT |
| | (SEQ ID NO: 95) | | (SEQ ID NO: 96) |

¹β-M: β2 microglobulin

### DETAILED DESCRIPTION

Genomic imprinting is an epigenetic process responsible for mono-allelic expression of the so-called imprinted genes (Reik & Walter (2001) Nat Rev Genet 2:21-32). There are at least 80 imprinted genes (i.e., expressed from maternal or paternal chromosomes only) that have been identified for which mono-allelic expression appears to be relevant to proper development (Yamazaki et al. (2003) Proc Natl Acad Sci USA 100:12207-12212; Pannetier & Feil (2007) Trends Biotechnol 25:556-562; Horii et al. (2008) Stem Cells 26:79-88). In addition, most imprinted genes such as insulin-like growth factor 2 (Ig12), H19, Igf2 receptor (Igf2R) and p57Kip2 (also known as Cdkn1c) have a direct role in embryo development (Reik & Walter (2001) Nat Rev Genet 2:21-32).

The majority of imprinted genes exist as gene clusters enriched for CpG islands and their expression is coordinately regulated by DNA methylation status on CpG-rich cis elements known as differentially methylated regions (DMRs). The DMRs are differentially methylated on CpG sites by DNA methyltransferase (Dnmts), depending on the parental allele origin (Delaval & Feil (2004) Curr Opin Genet Dev 14:188-195). In addition, depending on the developmental period of methylation, “primary DMRs” are differentially methylated during gametogenesis, and “secondary DMRs” acquire allele-specific methylation after fertilization (Lopes at al. (2003) Hum Mol Genet 12:295-305). So far, 15 primary DMRs have been identified in the mouse genome. Interestingly, most DMRs are methylated in the maternal allele and only three DMRs (Igf2-H19, Rasgrf1, Meg3 loci) are paternally methylated (Kobayashi at al. (2006) Cytogenet Genome Res 113:130-137). Although DMR methylation is of primary importance, histone modifications also contribute to monoallelic expression of these genes (Fournier at al. (2002) EMBO J 21:6560-6570; Mager at al. (2003) Nat Genet 33:502-507).

Recently, a population of very small embryonic like (VSEL) stem cells (SCs) was identified in adult bone marrow (BM; see PCT International Patent Application Publication Nos. WO 2007/067280 and 2009/059032, the entire disclosures of which are incorporated herein by reference). These VSELs: (i) are very small in size (about 3-6 μm); (ii) are positive for Oct-4, CXCR4, SSEA-1, and Sca-1; (iii) are CD45 negative and lineage negative; iv) possess large nuclei containing unorganized chromatin (euchromatin); and v) form embryoid body-like spheres (VSEL-DSs) that contain primitive SCs that are capable of differentiating into cell types derived from all three germ layers when co-cultured with C2C12 cells. Unlike ES cells, however, highly purified BM-derived Oct4+ VSELs do not proliferate in vitro if cultured alone, and do not grow teratomas in vivo. In co-cultures with myoblastic C2C12 cells, VSELs form embryoid body-(EB) like structures, referred to herein as VSEL-derived spheres (VSEL-DSs), which contain primitive stem cells able to differentiate into cells from all three germ layers (Kucia at al. (2006a) Leukemia 20:857-869). On the one hand, this suggests that VSELs are a quiescent cell population and that mechanisms must exist to prevent their unleashed proliferation and teratoma formation. On the other hand, the ability of VSELs to change their quiescent fate in co-cultures with C2C12 cells shows that their quiescent status can be modulated. This supports the concept that VSELs can contribute to rejuvenation of organs and tissue repair.

Disclosed herein is the discovery that imprinting status differs among various types of pluripotent cells at several imprinted genes, and that these differences can be exploited to prepare subpopulations of pluripotent cells for use in cell replacement therapies, among other uses. Also disclosed herein is the discovery that the proliferative quiescence of VSELs can be epigenetically controlled by DNA methylation on developmentally important imprinted genes.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent present in the pharmaceutical composition. It is noted, however, that carriers, excipients, and other inactive agents can and likely would be present in the pharmaceutical composition.

With respect to the terms “comprising”, “consisting of”, and “consisting essentially of”, where one of these three terms is used herein, the presently disclosed and claimed subject matter can include the use of either of the other two terms. For example, in some embodiments, a kit of the presently disclosed subject matter comprises a plurality of oligonucleotide primers. It would be understood by one of ordinary skill in the art after review of the instant disclosure that the presently disclosed subject matter also encompasses a kit that consists essentially of the same or a different plurality of oligonucleotide primers, as well as consists of the same or a different plurality of oligonucleotide primers.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes listed in Tables 1 and 2, which disclose GENBANK® Accession Nos. for the murine and human nucleic acid sequences, respectively, are intended to encompass homologous genes and gene products from other animals including, but not limited to other mammals, fish, amphibians, reptiles, and birds.

The methods of the presently disclosed subject matter are particularly useful for warm-blooded vertebrates. Thus, the presently disclosed subject matter concerns mammals and birds. More particularly contemplated is the isolation, manipulation, and use of VSEL stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of VSEL stem cells from livestock, including but not limited to domesticated swine (pigs and hogs), ruminants, horses, poultry, and the like.

The term “about”, as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods.

The term “isolated”, as used in the context of a nucleic acid or polypeptide (including, for example, a peptide), indicates that the nucleic acid or polypeptide exists apart from its native environment. An isolated nucleic acid or polypeptide can exist in a purified form or can exist in a non-native environment.

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid.

The terms “nucleic acid molecule” and “nucleic acid” can also be used in place of “gene”, “cDNA”, and “mRNA”. Nucleic acids can be synthesized, or can be derived from any biological source, including any organism.

The term “isolated”, as used in the context of a cell (including, for example, a VSEL stem cell), indicates that the cell exists apart from its native environment. An isolated cell can also exist in a purified form or can exist in a non-native environment.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

### II. Methods for Determining Degree of Pluripotency

In some embodiments, the presently disclosed subject matter provides methods for determining degrees of pluripotency amongst cell populations. As used herein, the phrase “degree of pluripotency” refers to a relative assessment of the pluripotency of a first cell with respect to a second cell or plurality of cells. As would be understood by one of ordinary skill in the art upon a review of the instant disclosure, the ability of a given cell to differentiate into different cell types in vitro or in vivo ranges from a completely unrestricted capacity (i.e., so-called “totipotent” cells) to no capacity for further differentiation (i.e., terminally differentiated cells).

Between these two extremes are cells that are characterized by different degrees of differentiative capacity, which are referred to as “pluripotent” cells. As used herein, a “pluripotent” cell is a cell that can differentiate into at least two different terminally differentiated cell types, and in some embodiments can differentiate into more than two different terminally differentiated cell types. In some embodiments, a pluripotent cell can also self-renew, meaning that when the cell divides, at least one of the daughter cells retains the same differentiative capacity as the parent cell (e.g., at least one of the daughter cells is also a pluripotent cell).

For example, embryonic stem (ES) cells, which have been shown in some mammals to have the potential to differentiate into all the different cell types of the animal, are pluripotent cells, and in some embodiments can also be considered totipotent cells. Primordial germ cells (PGCs) can also be manipulated in culture to form embryonic germ cells (see U.S. Pat. Nos. 5,453,357; 5,670,372; 5,690,926; and 7,153,684), which appear to have the same differentiative capacity as ES cells, and thus are also pluripotent and possibly totipotent.

Certain other stem cells, by contrast, have, lost the ability to differentiate into at least some cell types. Exemplary such stem cells, which are nonetheless pluripotent, include hematopoietic stem cells (HSCs), mesenchymal stem cells (MSCs), and multipotent adult progenitor cells (MAPCs), to name a few. These stem cell types are considered to be less pluripotent than VSELs due to the limited repertoire of cell types into which the former (or daughter cells therefrom) can differentiate as compared to VSELs.

In some embodiments, the methods of the presently disclosed subject matter assess relative degrees of pluripotency among stem cell types by comparing imprinting statuses of selected loci among the various stem cell types. As used herein, the phrase “imprinting status” refers to a degree of methylation of one or more regions of a locus that has been shown to be imprinted.

As used herein, the term “imprinted” and grammatical variants thereof refers to a genetic locus for which one of the parental alleles is repressed and the other one is transcribed and expressed, and the repression or expression of the allele depends on whether the genetic locus was maternally or paternally inherited. Thus, an imprinted genetic locus can be characterized by parent-of-origin dependent monoallelic expression: the two alleles present in an individual are subject to a mechanism of transcriptional regulation that is dependent on which parent transmitted the allele. Imprinting can be species- and tissue-specific as well as a developmental-stage-specific phenomenon (see e.g., Weber et al. (2001) Mech Devel 101:133-141; Murphy & Jirtle (2003) Bioessays 25:577-588).

At least 80 loci have been found to be imprinted in mammals (see Morison et al. (2005) Trends Genet 21:457-465). As disclosed herein, several of these loci have been found to be differentially imprinted in VSELs versus other stem cell types. These loci include, but are not limited to the Igf2/H19 locus, the Rasgrf1 locus, the Igf2R locus, the Kcnq1 locus, Peg1/Mest locus, the Meg3 locus, the p57^{KIP2} locus, the p21^{Cip1} locus, the p18^{INK4c} locus, and the SNRPN locus.

As used herein, the term “Igf2” refers to insulin-like growth factor 2 (somatomedin A), which corresponds to GENBANK® Accession Nos. NC_{—}000011 (genomic sequence from human chromosome 11, nucleotides 2,150,347 to 2,170,833), NM_{—}000612 (transcript variant 1 cDNA sequence), and NP_{—}000603.1 (amino acid sequence encoded by the transcript variant 1 cDNA sequence). The Igf2 locus has been shown to be imprinted, with the maternal allele being methylated (see Kobayashi et al. (2006) Genome Res 113:130-137).

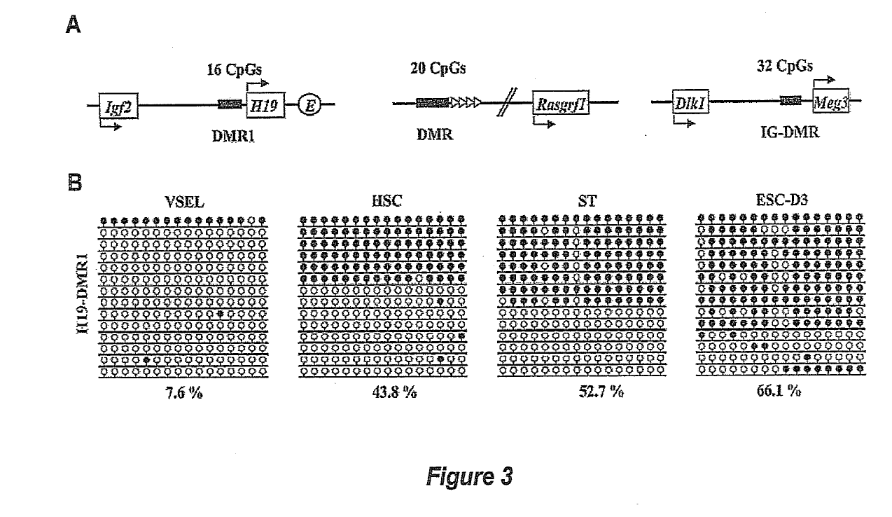

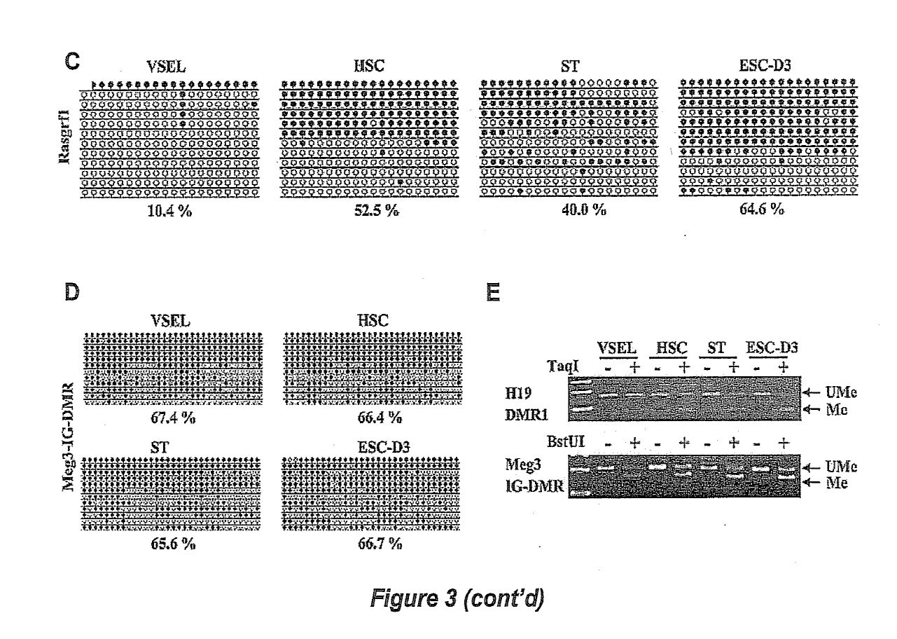

As used herein, the term “H19” refers to H19, which is an imprinted, maternally-expressed but non-protein coding RNA that corresponds to GENBANK® Accession Nos. NC_{—}000011 (genomic sequence from human chromosome 11, nucleotides 2,016,406 to 2,019,065) and NR_{—}002196 (cDNA sequence). The H19 locus is located on human chromosome 11 in the vicinity of the insulin-like growth factor 2 (IGF2) locus. The H19 locus is expressed from the maternally-inherited chromosome, whereas the Igf2 locus is expressed from the paternally-inherited chromosome. There is a differentially-methylated region (DMR) referred to as “DMR1” located between the promoters for Igf2 and H19 (see FIG. 3A), and as disclosed herein, methylation differences between VSELs and other cell types were identified at DMR1.

As used herein, the term “Rasgrf1” refers to Ras protein-specific guanine nucleotide-releasing factor 1. This locus corresponds to GENBANK® Accession Nos. NC_{—}000015 (genomic sequence from human chromosome 15, nucleotides 79,252,289 to 79,383,215), NM_{—}002891 (nucleotide sequence of the transcript variant 1 cDNA), and NP_{—}002882 (amino acid sequence encoded by NM_{—}002891). The Rasgrf1 locus has been shown to be imprinted by paternal allele methylation at a DMR located 30 kilbase pairs 5′ of its promoter (Yoon et al. (2005) Mol Cell Biol 25:11184-11190).

As used herein, the term “Igf2R” refers to the insulin-like growth factor 2 receptor, the locus for which corresponds to GENBANK® Accession Nos. NC_{—}000006 (genomic sequence from human chromosome 6, nucleotides 160,390,131 to 160,527,583), NM_{—}000876 (nucleotide sequence of a cDNA derived from this locus), and NP_{—}000867 (amino acid sequence encoded by NM_{—}000876). The Igfr2 locus has been shown to be imprinted, wherein in most tissues, expression from the paternal allele is suppressed by methylation while the maternal allele is completely unmethylated and expressed. In the central nervous system, however, both parental alleles are unmethylated and expressed (see Hu et al. (1998) Mol Endocrinol 12:220-232).

As used herein, the term “Kcnq1” refers to potassium voltage-gated channel, KQT-like subfamily, member 1, the locus for which corresponds to GENBANK® Accession Nos. NC_{—}000011 (genomic sequence from human chromosome 11, nucleotides 2,466,221 to 2,870,340), NM_{—}000218 (transcript variant 1 cDNA sequence), and NP_{—}000209 (amino acid sequence encoded by NM_{—}000218). An imprint control region (ICR) has been identified in intron 10 of the human Kcnq1 gene (Thakur et al. (2004) Mol Cell Biol 24:7855-7862).

As used herein, the term “Peg1/Mest” refers to paternally-expressed gene 1/mesoderm specific transcript homolog (mouse), which is a locus on human chromosome 7 that corresponds to GENBANK® Accession Nos. NC_{—}000007 (nucleotides 130,126,046 to 130,146,133), NM_{—}002402 (transcript variant 1 cDNA sequence), and NP_{—}002393 (amino acid sequence encoded by NM_{—}002402). The Peg1/Mest locus has been shown to be maternally-imprinted, resulting in only the paternally-inherited allele being active in all tissues tested in mice and in humans (Reule et al. (1998) Dev Genes Evol 208:161-163).

As used herein, the term “Meg3” refers to maternally expressed 3, a non-protein-encoding locus on human chromosome 14 that corresponds to GENBANK® Accession Nos. NC_{—}000014 (nucleotides 101,292,445 to 101,327,368) and NR_{—}002766. Meg3 is a maternally-expressed imprinted gene, and alternative splicing results in several transcript variants being produced from this locus (Miyoshi et al. (2000) Genes Cells 5:211-220).

As used herein, the terms “p57^{KIP2}” and “CDKN1C” refer to cyclin-dependent kinase inhibitor 1C (p57, Kip2), a locus on human chromosome that corresponds to GENBANK® Accession Nos. NC_{—}0000011 (nucleotides 2,904,448 to 2,906,995), NM_{—}000076 (transcript variant 1 cDNA sequence), and NP_{—} 000067 (amino acid sequence encoded by NM_{—}000076). See Lee et al. (1995) Genes Dev 9:639-649. At least three transcript variants and 2 isoforms have been identified for this maternally-expressed imprinted locus. Mutations in the locus have also been associated with Beckwith-Wiedemann syndrome, suggesting that the p57^{KIP2} locus might encode a tumor suppressor. (Hatada et al. (1996) Nat Genet 14:171-173).

As used herein, the terms “p21^{Cip1}” and “CDKN1A” refer to cyclin-dependent kinase inhibitor 1A (p21, Cip1), which is a locus on human chromosome 6 that corresponds to GENBANK® Accession Nos. NC_{—}000006 (nucleotides 36,646,459 to 36,655,109), NM_{—}000389 (transcript variant 1 cDNA sequence), and NP_{—}000380 (amino acid sequence encoded by NM_{—}000389). See Demetrick at al. (1995) Cytogenet. Cell Genet 69:190-192. The p21^{Cip1} locus is a maternally-expressed imprinted locus.

As used herein, the terms “p18^{INK4c}” and “CDKN2C” refer to cyclin-dependent kinase inhibitor 2C (p18, inhibits CDK4), which is a locus on human chromosome 1 that corresponds to GENBANK® Accession Nos. NC_{—}000001 (nucleotides 51,434,367 to 51,440,309), NM_{—}001262 (transcript variant 1 cDNA sequence), and NP_{—}001253 (amino acid sequence encoded by NM_{—}001262). See Serrano et al. (1993) Nature 366:704-707.

As used herein, the term “SNRPN” refers to small nuclear ribonucleoprotein polypeptide N, which is a locus on human chromosome 15 that corresponds to GENBANK® Accession Nos. NC_{—}0000015 (nucleotides 25,068,794 to 25,664,609), NM_{—}003097 (transcript variant 1 cDNA sequence), and NP_{—}003088 (amino acid sequence encoded by NM_{—}003097). The SNRPN is maternally imprinted and has been found to be deleted in Prader-Willi syndrome (Reed & Leff (1994) Nat Genet 6:163-167).

It is noted that with respect to all nucleotide sequences of genetic loci disclosed herein, where a single transcript variant is disclosed, all other transcript variants that are present in the GENBANK® database are also included within the scope of the instant disclosure.

Various tests that are known to one of ordinary skill in the art can identify and/or assay for the imprinting status of these and other imprinted genes. For example, methylation profiles (i.e., a summary of the methylation. status(es) of one or more loci in a cell or cell type) can be detected by simple hybridization analysis (e.g., Southern blotting) of nucleic acids cleaved with methyl-sensitive or methyl-dependent restriction endonucleases to detect methylation patterns. Typically, these methods involve use of one or more targets that hybridize to at least one sequence that may be methylated. The presence or absence of methylation of a restriction sequence is determined by the length of the polynucleotide hybridizing to the probe. This and other methods for detecting DNA methylation are described in, e.g., Thomassin et al. (1999) Methods 19:465-475 and U.S. Pat. No. 7,186,512.

One such method is bisulfite sequencing (see also Warnecke et al. (1990) Genomics 51:182-190). The phrase “bisulfite sequencing” refers to the use of bisulfite to modify DNA following by sequencing of the modified DNA to determine the methylation pattern of the (unmodified) DNA. Bisulfite sequencing takes advantage of the addition of a methyl group to the carbon-5 position of cytosine residues present within the dinucleotide CpG. Treatment of DNA with bisulfite converts unmodified cytosines to uracil, whereas 5-methylcytosine residues are unaffected. As a consequence, treatment with bisulfite introduces specific sequence changes in DNA molecules that result from the methylation statuses of cytosine residues present therein. Sequencing of nucleic acids that have been treated with bisulfite (i.e., “bisulfite sequencing”) can then be used to determine the overall methylation status of the nucleic acid by comparing the sequence identified with a standard sequence (i.e., the same nucleic acid sequenced without bisulfite treatment).

Other strategies can also be employed to determine the methylation patterns at loci of interest subsequent to bisulfite treatment. Exemplary such methods include restriction analysis using endonucleases that differentially restrict DNA based on differences in methylation (see e.g., Sadri et al. (1996) Nucleic Acids Res (1996) 24:4987-4989).

Another technique that can be employed to identify the methylation status of a nucleic acid is the and combined bisulfite-restriction analysis (COBRA) technique (Xiong & Laird (1997) Nucleic Acids Res 25:2532-2534). In this method, standard bisulfite treatment is used to introduce methylation-dependent sequence differences into a nucleic acid (for example, a subsequence of a genomic DNA). The nucleic acid (or a subsequence thereof) is then PCR amplified using primers that flank the sequence to be assayed. The bisulfite treatment results in the PCR amplification products having sequences that reflect the presence or absence of methylated-cytosines in the original nucleic acid molecule. Any sequence changes that result can lead to the methylation-dependent creation of new restriction enzyme sites or it can lead to the methylation-dependent retention of pre-existing sites such as. The products of the PCR reaction are then digested with appropriate restriction enzymes, and the products of the digestion reactions are visualized. Based on the sizes of the digestion products, it is possible to determine the methylation statuses of known sequences presented in the original nucleic acid molecule.

Carrier Chromatin-Immunoprecipitation (Carrier-ChIP; O'Neill et al. (2006) Nat Genet 38:835-841) can also be employed to assay DNA methylation. A kit for performing this assay is commercially available (MAGNA CHIP™ G kit, Upstate-Millipore, Billerica, Mass., United States of America).

Using any of these exemplary techniques, either alone or in combination, the methylation statuses of different cell preparations (e.g., preparations of VSELs or other cell types of interest including, but not limited to other types of stem cells) can be determined. After methylation statuses are determined, they can be compared to identify how they differ among different cell types (e.g., stem cell types). For example, the methylation statuses of various loci of exemplary totipotent cells such as ES cells can be compared to the methylation statuses of the same loci in more differentiated (i.e., less pluripotent) cells such as HSCs, bone marrow mononuclear cells (BMMNCs), and/or MSCs. Given the relative levels of pluripotency of these cell lines, methylation profiles for these cell types can be established and compared to the methylation profiles of cell types of interest such as, but not limited to VSELs.

As disclosed herein, it has been determined that relative to the methylation profile a first cell type of interest, a methylation profile of a second cell type of interest that is characterized by hypomethylation at the Igf2-H19 locus, hypomethylation at the Rasgrf1 locus, hypermethylation at the Igf2R locus, hypermethylation at the Kcnq1 locus, and/or hypermethylation at the Peg1/Mest locus is indicative of the second cell type being in a more pluripotent state than the first cell type. Similarly, a methylation profile of a second cell type of interest that is characterized by hypermethylation at the Igf2-H19 locus, hypermethylation at the Rasgrf1 locus, hypomethylation at the Igf2R locus, hypomethylation at the Kcnq1 locus, and/or hypomethylation at the Peg1/Mest locus is indicative of the second cell type being in a less pluripotent state than the first cell type.

III. Method for Distinguishing VSELs from Other Stem Cells

The presently disclosed subject matter also provides methods for distinguishing VSELs from other stem cell types including, but not limited to hematopoietic stem cells (HSCs) and mesenchymal stem cells (MSCs). This can be accomplished by comparing methylation profiles between VSELs and other stem cell types of interest. When a profile is established for VSELs and the other stem cell types of interest, differences between the profiles can be employed for distinguishing VSELs from these other cell types.

For example, the presently disclosed methods can comprise comparing a methylation profile comprising imprinting statuses of one or more loci of the VSEL selected from the group consisting of Igf2-H19, Rasgrf1, Igf2R, Kcnq1, and Peg1/Mest to the same one or more loci in the second cell type(s) of interest (e.g., an HSC or an MSC), wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus in the VSEL relative to the levels of methylation at these same loci in the other cell type(s) (e.g., the HSC or the MSC) are indicative of VSELs.

IV. Methods for Isolating VSELs from Sources Expected to Contain VSELs

The presently disclosed subject matter also provides methods for isolating VSELs from sources expected to contain VSELs. In some embodiments, the methods comprise isolating a plurality of CD45^{neg}/lin^{neg} cells that are Sca-1⁺ or CD34⁺ from the source; and isolating a subset of cells from the plurality of CD45^{neg}/lin^{neg} cells that are Sca-1⁺ or CD34⁺, wherein the subset of cells are characterized by one or more of hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus relative to the fraction of cells present in the plurality of CD45^{neg}/lin^{neg} cells that are Sca-1⁺ or CD34⁺ from the source that are not isolated in this step. In some embodiments, the methods can further comprise fractionating the cells to identify cells that are Oct-4⁺, CXCR4, and/or SSEA-1⁺.

As used herein, the term “CD45” refers to a tyrosine phosphatase, also known as the leukocyte common antigen (LCA), and having the gene symbol PTPRC. This gene corresponds to GENBANK® Accession Nos. NP_{—}002829 (human), NP_{—}035340 (mouse), NP_{—}612516 (rat), XP_{—}002829 (dog), XP_{—}599431 (cow) and AAR16420 (pig). The amino acid sequences of additional CD45 homologs are also present in the GENBANK® database, including those from several fish species and several non-human primates.

As used herein, the term “CD34” refers to a cell surface marker found on certain hematopoietic and non-hematopoietic stem cells, and having the gene symbol CD34. The GENBANK® database discloses amino acid and nucleic acid sequences of CD34 from humans (e.g., AAB25223), mice (NP_{—}598415), rats (XP_{—}223083), cats (NP_{—}001009318), pigs (MP_{—}999251), cows (NP_{—}776434), and others.

In mice, some stem cells also express the stem cell antigen Sca-1 (GENBANK® Accession No. NP_{—}034868), also referred to as Lymphocyte antigen Ly-6A.2.

Thus, the subpopulation of CD45^{neg} stem cells represents a subpopulation of all CD45^{neg} cells that are present in the population of cells prior to the separating step. In some embodiments, the cells of the subpopulation of CD45^{neg} stem cells are from a human, and are CD34⁺/lin^{neg}/CD45^{neg}. In some embodiments, the cells of the subpopulation of CD45^{neg} stem cells are from a mouse, and are Sca-1⁺/lin^{neg}/CD45^{neg}.

The isolation of the disclosed subpopulations can be performed using any methodology that can separate cells based on expression or lack of expression of the one or more of the CD45, CXCR4, CD34, AC133, Sca-1, CD45R/B220, Gr-1, TCRaβ, TCRγδ, CD11b, and Ter-119 markers including, but not limited to fluorescence-activated cell sorting (FACS).

As used herein, lin^{neg} refers to a cell that does not express any of the following markers: CD45R/B220, Gr-1, TCRaβ, TCRγδ, CD11b, and Ter-119. These markers are found on cells of the B cell lineage from early Pro-B to mature B cells (CD45R/B220); cells of the myeloid lineage such as monocytes during development in the bone marrow, bone marrow granulocytes, and peripheral neutrophils (Gr-1); thymocytes, peripheral T cells, and intestinal intraepithelial lymphocytes (TCRaβ and TCRγδ); myeloid cells, NK cells, some activated lymphocytes, macrophages, granulocytes, B1 cells, and a subset of dendritic cells (CD11b); and mature erythrocytes and erythroid precursor cells (Ter-119).

The separation step can be performed in a stepwise manner as a series of steps or concurrently. For example, the presence or absence of each marker can be assessed individually, producing two subpopulations at each step based on whether the individual marker is present. Thereafter, the subpopulation of interest can be selected and further divided based on the presence or absence of the next marker.

Alternatively, the subpopulation can be generated by separating out only those cells that have a particular marker profile, wherein the phrase “marker profile” refers to a summary of the presence or absence of two or more markers. For example, a mixed population of cells can contain both CD34⁺ and CD34^{neg} cells. Similarly, the same mixed population of cells can contain both CD45⁺ and CD45^{neg} cells. Thus, certain of these cells will be CD34⁺/CD45⁺, others will be CD34⁺/CD45^{neg}, others will be CD34^{neg}/CD45⁺, and others will be CD34^{neg}/CD45^{neg}. Each of these individual combinations of markers represents a different marker profile. As additional markers are added, the profiles can become more complex and correspond to a smaller and smaller percentage of the original mixed population of cells. In some embodiments, the cells of the presently disclosed subject matter have a marker profile of CD34⁺/lin^{neg}/CD45^{neg}, and in some embodiments, the cells of the presently disclosed subject matter have a marker profile of Sca-1⁺/lin^{neg}/CD45^{neg}.

In some embodiments of the presently disclosed subject matter, antibodies specific for markers expressed by a cell type of interest (e.g., polypeptides expressed on the surface of a CD34⁺/lin^{neg}g/CD45^{neg} or a Sca-1⁺/lin^{neg}/CD45^{neg} cell) are employed for isolation and/or purification of subpopulations of BM cells that have marker profiles of interest. It is understood that based on the marker profile of interest, the antibodies can be used to positively or negatively select fractions of a population, which in some embodiments are then further fractionated.

In some embodiments, a plurality of antibodies, antibody derivatives, and/or antibody fragments with different specificities is employed. In some embodiments, each antibody, or fragment or derivative thereof, is specific for a marker selected from the group including but not limited to Ly-6A/E (Sca-1), CD34, CXCR4, AC133, CD45, CD45R, B220, Gr-1, TCRαβ, TCRγδ, CD11b, Ter-119, c-met, LIF-R, SSEA-1, Oct-4, Rev-1, and Nanog. In some embodiments, cells that express one or more genes selected from the group including but not limited to SSEA-1, Oct-4, Rev-1, and Nanog are isolated and/or purified.

The presently disclosed subject matter relates to a population of cells that in some embodiments express the following antigens: CXCR4, AC133, CD34, SSEA-1 (mouse) or SSEA-4 (human), fetal alkaline phosphatase (AP), c-met, and the LIF-Receptor (LIF-R). In some embodiments, the cells of the presently disclosed subject matter do not express the following antigens: CD45, Lineage markers (i.e., the cells are lin^{neg}), HLA-DR, MHC class I, CD90, CD29, and CD105. Thus, in some embodiments the cells of the presently disclosed subject matter can be characterized as follows: CXCR4⁺/AC133⁺/CD34⁺/SSEA-1⁺ (mouse) or SSEA-4⁺ (human)/AP⁺/c-met⁺/LIF-R⁺/CD45^{neg}/lin^{neg}/HLA-DR^{neg}/MHC class I^{neg}/CD90^{neg}/CD29^{neg}/CD105^{neg}.

In some embodiments, each antibody, or fragment or derivative thereof, comprises a detectable label. Different antibodies, or fragments or derivatives thereof, which bind to different markers can comprise different detectable labels or can employ the same detectable label.

A variety of detectable labels are known to the skilled artisan, as are methods for conjugating the detectable labels to biomolecules such as antibodies and fragments and/or derivatives thereof. As used herein, the phrase “detectable label” refers to any moiety that can be added to an antibody, or a fragment or derivative thereof, that allows for the detection of the antibody. Representative detectable moieties include, but are not limited to, covalently attached chromophores, fluorescent moieties, enzymes, antigens, groups with specific reactivity, chemiluminescent moieties, and electrochemically detectable moieties, etc. In some embodiments, the antibodies are biotinylated. In some embodiments, the biotinylated antibodies are detected using a secondary antibody that comprises an avidin or streptavidin group and is also conjugated to a fluorescent label including, but not limited to Cy3, Cy5, and Cy7. In some embodiments, the antibody, fragment, or derivative thereof is directly labeled with a fluorescent label such as Cy3, Cy5, or Cy7. In some embodiments, the antibodies comprise biotin-conjugated rat anti-mouse Ly-6A/E (Sca-1; clone E13-161.7), streptavidin-PE-Cy5 conjugate, anti-CD45-APCCy7 (clone 30-F11), anti-CD45R/B220-PE (clone RA3-6B2), anti-Gr-1-PE (clone RB6-8C5), anti-TCRαβ PE (clone H57-597), anti-TCRγδ PE (clone GL3), anti-CD11b PE (clone M1/70) and anti-Ter-119 PE (clone TER-119). In some embodiments, the antibody, fragment, or derivative thereof is directly labeled with a fluorescent label and cells that bind to the antibody are separated by fluorescence-activated cell sorting. Additional detection strategies are known to the skilled artisan.

While FACS scanning is a convenient method for purifying subpopulations of cells, it is understood that other methods can also be employed. An exemplary method that can be used is to employ antibodies that specifically bind to one or more of CD45, CXCR4, CD34, AC133, Sca-1, CD45R/B220, Gr-1, TCRaβ, TCRγδ, CD11b, and Ter-119, with the antibodies comprising a moiety (e.g., biotin) for which a high affinity binding reagent is available (e.g., avidin or streptavidin). For example, a biotin moiety could be attached to antibodies for each marker for which the presence on the cell surface is desirable (e.g., CD34, Sca-1, CXCR4), and the cell population with bound antibodies could be contacted with an affinity reagent comprising an avidin or streptavidin moiety (e.g., a column comprising avidin or streptavidin). Those cells that bound to the column would be recovered and further fractionated as desired. Alternatively, the antibodies that bind to markers present on those cells in the population that are to be removed (e.g., CD45R/B220, Gr-1, TCRaβ, TCRγδ, CD11b, and Ter-119) can be labeled with biotin, and the cells that do not bind to the affinity reagent can be recovered and purified further.

It is also understood that different separation techniques (e.g., affinity purification and FACS) can be employed together at one or more steps of the purification process.

A population of cells containing the CD34⁺/lin^{neg}/CD45^{neg} or Sca-1⁺/lin^{neg}/CD45^{neg} cells of the presently disclosed subject matter can be isolated from any subject or from any source within a subject that contains them. In some embodiments, the population of cells comprises a bone marrow sample, a cord blood sample, or a peripheral blood sample. In some embodiments, the population of cells is isolated from peripheral blood of a subject subsequent to treating the subject with an amount of a mobilizing agent sufficient to mobilize the CD45^{neg} stem cells from bone marrow into the peripheral blood of the subject. As used herein, the phrase “mobilizing agent” refers to a compound (e.g., a peptide, polypeptide, small molecule, or other agent) that when administered to a subject results in the mobilization of a VSEL stem cell or a derivative thereof from the bone marrow of the subject to the peripheral blood. Stated another way, administration of a mobilizing agent to a subject results in the presence in the subject's peripheral blood of an increased number of VSEL stem cells and/or VSEL stem cell derivatives than were present therein immediately prior to the administration of the mobilizing agent. It is understood, however, that the effect of the mobilizing agent need not be instantaneous, and typically involves a lag time during which the mobilizing agent acts on a tissue or cell type in the subject in order to produce its effect. In some embodiments, the mobilizing agent comprises at least one of granulocyte-colony stimulating factor (G-CSF) and a CXCR4 antagonist (e.g., a T140 peptide; Tamamura at al. (1998) 253 Biochem Biophys Res Comm 877-882).

In some embodiments, a VSEL stem cell or derivative thereof also expresses a marker selected from the group including but not limited to c-met, c-kit, LIF-R, and combinations thereof. In some embodiments, the disclosed isolation methods further comprise isolating those cells that are c-met⁺, c-kit⁺, and/or LIF-R⁺.

In some embodiments, the VSEL stem cell or derivative thereof also expresses SSEA-1, Oct-4, Rev-1, and Nanog, and in some embodiments, the disclosed isolation methods further comprise isolating those cells that express these genes.

The presently disclosed subject matter also provides a population of CD45^{neg} stem cells isolated by the presently disclosed methods.

### V. Compositions for Analyzing Methylation Patterns in Cells

The presently disclosed subject matter also provides kits that can be employed in the practice of the disclosed methods. In some embodiments, the kits comprise a plurality of oligonucleotide primers, wherein the oligonucleotide primers specifically bind to a subsequence of a differentially methylated region (DMR) in a nucleic acid or bind to a nucleotide sequence that flanks a DMR in a nucleic acid, wherein the oligonucleotide primers can be used to assay the methylation status of at least one methylated nucleotide present within the DMR. In some embodiments, the DMR is a human DMR and the oligonucleotide primers specifically bind to a subsequence of the human genome that comprises the DMR or that specifically bind to a subsequence of the human genome that comprises the DMR only, after the subsequence of the human genome comprising the DMR has been treated with bisulfite.

As used herein, the phrase “specifically binds” refers to an oligonucleotide that only binds to a region of a nucleic acid to be assayed (e.g., a subsequence of a genomic DNA that comprises a DMR the methylation status of which is of interest) and does not bind to other regions of other nucleic acids that might also be present. In this way, an oligonucleotide primer that specifically binds to a subsequence of a differentially methylated region (DMR) in a nucleic acid or that binds to a nucleotide sequence that flanks a DMR in a nucleic acid can be used to assay the methylation status of at least one methylated nucleotide present within the DMR. Representative oligonucleotide primers include those disclosed herein as SEQ ID NOs: 1-96, although it is understood that other oligonucleotide primers can be designed that can be used to assay the methylation profiles of the imprinted loci disclosed herein taking into account the sequences of the loci present in, for example, the GENBANK® database.

As such, the presently disclosed kits can comprise oligonucleotides that specifically bind to a DMR is present in an Igf2-H19 locus, a Rasgrf1 locus, an Igf2R locus, a Kcnq1 locus, or a Peg1/Mest locus, as well as combinations of such oligonucleotides. As would be understood by one of ordinary skill in the art after review of the instant disclosure, the plurality of oligonucleotides present in the kit can also include pairs of oligonucleotides that are designed to be used together to assay these or other imprinted loci. For example, the oligonucleotides set forth in Tables 1 and 2 hereinabove can be used in pairs or pluralities of pairs to assay any of the Igf2-H19, Rasgrf1, Igf2R, Kcnq1, and/or Peg1/Mest loci.

Furthermore, the kits can include oligonucleotides that can be employed in techniques including, but not limited to bisulfite sequencing, carrier chromatin-immunoprecipitation (ChIP), and quantitative ChIP (qChIP).

### VI. Methods for Assessing the Purity of a VSEL Preparation

In some embodiments, the presently disclosed subject matter provides methods for assessing the purity of a very small embryonic like stem cell (VSEL) preparation. In some embodiments, the methods comprise providing a first preparation suspected of comprising VSELs; and comparing an imprinting profile of cells of the first preparation with respect to one or more loci selected from the group consisting of Igf2-H19, Rasgrf1, Igf2R, Kcnq1, and Peg1/Mest to an imprinting profile of a second preparation of VSELs with respect to the same one or more loci, wherein relative to the second preparation, hypermethylation at the Igf2-H19 locus, hypermethylation at the Rasgrf1 locus, hypomethylation at the Igf2R locus, hypomethylation at the Kcnq1 locus, and hypomethylation at the Peg1/Mest locus relative to levels of methylation at these loci in the second preparation is indicative of the first preparation being less pure with respect to VSELs than the second preparation. An imprinting status of the preparations at other imprinted loci including, but not limited to Meg3, p57^{KIP2}, p21^{Cip1}, p18^{INK4c}, and SNRPN can also be included within the imprinting profile.

The first and second preparations can be isolated from any source that is expected to contain VSELs. Exemplary sources include bone marrow, cord blood, fetal liver, and adult tissues. In some embodiments, the first preparation is isolated from a source that includes other stem cells such as, but not limited to HSCs and MSCs, and the purity of the first preparation with respect to VSELs is assessed relative to the HSC content and/or the MSC context of the first preparation.

In some embodiments, the second preparation is a preparation that is highly purified for VSELs. As used herein, the phrase “highly purified” refers to a preparation that comprises in some embodiments at least 50% VSELs, in some embodiments at least 60% VSELs, in some embodiments at least 70% VSELs, in some embodiments at least 75% VSELs, in some embodiments at least 80% VSELs, in some embodiments at least 85% VSELs, in some embodiments at least 90% VSELs, in some embodiments at least 95% VSELs, in some embodiments at least 97% VSELs, and in some embodiments at least 99% VSELs. By comparing an imprinting profile of the first preparation to the second preparation (e.g., a profile comprising the imprinting status of at least one locus selected from the group consisting of Igf2-H19, Rasgrf1, Igf2R, Kcnq1, Peg1/Mest, Meg3, p57^{KIP2}, p21^{Cip1}, p18^{INK4c}, and SNRPN), the purity of the first preparation with respect to VSELs can be determined.

VII. Identification of Modulators of Imprinting in CD34⁺/lin^{neg}/CD45^{neg} or Sca-1⁺/lin^{neg}/CD45^{neg} Cells

The presently disclosed subject matter also relates to methods and compositions, for screening for modulators of imprinting in the CD34⁺/lin^{neg}/CD45^{neg} or Sca-1⁺/lin^{neg}/CD45^{neg} cells of the presently disclosed subject matter. As set forth herein, the ability of the CD34⁺/lin^{neg}/CD45^{neg} or Sca-1⁺/lin^{neg}/CD45^{neg} cells of the presently disclosed subject matter to change their quiescent fate in co-cultures with C2C12 cells shows that their quiescent status can be modulated. This supports the concept that the imprinting status of genetic loci present within the CD34⁺/lin^{neg}/CD45^{neg} or Sca-1⁺/lin^{neg}/CD45^{neg} cells of the presently disclosed subject matter can be altered in vitro and/or in vivo.

As such, in some embodiments the presently disclosed subject matter provides methods and combinations that can be employed to screen for a modulator of imprinting. As used herein, the phrase “modulator of imprinting” refers to a molecule (e.g., a biomolecule including, but not limited to a polypeptide, a peptide, or a lipid) that induces a change in the imprinting status of at least one locus (e.g., a locus selected from among Igf2-H19, Rasgrf1, Igf2R, Kcnq1, Peg1/Mest, Meg3, p57^{KIP2}, p21^{Cip1}, p18^{INK4c}and SNRPN) within a cell (e.g., a VSEL of the presently disclosed subject matter).

For example, co-culturing the CD34⁺/lin^{neg}/CD45^{neg} or Sca-1⁺/lin^{neg}/CD45^{neg} cells of the presently disclosed subject matter with C2C12 cells induces the CD34⁺/lin^{neg}/CD45^{neg} or Sca-1⁺/lin^{neg} /CD45^{neg} cells of the presently disclosed subject matter to differentiate into different cell types from all three embryonic germ layers (i.e., endoderm, mesoderm, and ectoderm). Thus, C2C12 cells produce at least one molecule (referred to herein as an “inducer”) that causes a change in the imprinting status of one or more loci in the CD34⁺/lin^{neg}/CD45^{neg} or Sca-1⁺/lin^{neg}/CD45^{neg} cells of the presently disclosed subject matter.

In some embodiments, the instant methods comprise (a) preparing a cDNA library comprising a plurality of cDNA clones from a cell known to comprise an inducer (e.g., C2C12 cells); (b) transforming a plurality of cells that do not comprise the inducer with the cDNA library; (c) culturing a plurality of CD34⁺/lin^{neg}/CD45^{neg} or Sca-1⁺/lin^{neg}/CD45^{neg} cells or derivatives thereof in the presence of the transformed plurality of cells under conditions sufficient to cause the CD34⁺/lin^{neg}/CD45^{neg} or Sca-1⁺/lin^{neg}/CD45^{neg} cells or derivatives thereof to form an embryoid body-like sphere; (d) isolating the transformed cell comprising the inducer; (e) recovering a cDNA clone from the transformed cell; and (f) identifying a polypeptide encoded by the cDNA clone recovered, whereby an inducer of embryoid body-like formation is identified. In some embodiments, the plurality of cDNA clones are present within a cDNA cloning vector, and the vector comprises at least one nucleotide sequence flanking at least one side of the cloning site in the vector into which the cDNA clones are inserted that can bind a primer such as a sequencing primer. In some embodiments, both primer-binding nucleotide sequences are present flanking each side of the cloning site, allowing the cDNA insert to be amplified using the polymerase chain reaction (PCR). Accordingly, in some embodiments the instant methods further comprise amplifying the cDNA clone present in the transformed cell using primers that hybridize to primer sites flanking both sides of the cDNA cloning site, and in some embodiments the identifying step is performed by sequencing the cDNA clone directly or by sequencing the amplified PCR product.

It is understood, however, that other methods that are within the skill of the ordinary artisan can also be employed to identify an inducer. For example, C2C12-conditioned medium can be tested to determine whether the inducer present in C2C12 cultures is a diffusible molecule (e.g., a peptide, polypeptide, or bioactive lipid). If the inducer is a diffusible molecule, the C2C12-conditioned medium can be heat treated to determine whether the inducer is heat labile (such as a peptide or polypeptide) or not heat labile (such as a bioactive lipid). Fractionation studies including, but not limited to proteomic analysis and/or lipid chromatography can then be employed to identify putative inducer.

If C2C12-conditioned medium does not comprise an inducer, it implies that the inducer is present on C2C12 cells. Techniques that can be applied for identifying a membrane-bound inducer that is present on C2C12 cells include, but are not limited to the use of monoclonal antibodies and/or siRNAs. Alternatively or in addition, gene expression analysis can be employed, including, for example, the use of gene arrays, differential display, etc.

When a putative inducer is identified, its status as an inducer can be confirmed by transforming a cell line that does not contain the inducer with a nucleotide sequence encoding the inducer and confirming that the transformed cell line supports the formation of embryoid body-like spheres by CD34⁺/lin^{neg}/CD45^{neg} or Sca-1⁺/lin^{neg}/CD45^{neg} cells or derivatives thereof.

Once potential inducers are identified, their abilities to induce imprinting changes at imprinted loci can be assessed. For example, certain types of tumors and cancers have been associated with changes in imprinting (see e.g., Holm et al. (2005) Cancer Cell 8:275-285). These cancers include human colorectal carcinogenesis related to aberrant expression of Igf2 (Cui et al. (2003) Science 299:1753-1755); oligodendrogliomas, breast cancer, and hepatocellular carcinomas (PEG3, P57, and IGF2R, respectively; De Souza et al. (1997) FASEB J 11:60-67; Kobatake et al. (2004) Oncol Rep 12:1087-1092; Trouillard et al. (2004) Cancer Genet Cytogenet 151:182-183); Prader-Willi syndrome (Reed & Leff (1994) Nat Genet 6:163-167); rhabdomyosarcoma (Casola et al. (1997) Oncogene 14:1503-1510; Anderson et al. (1999) Neoplasia 1: 340-348); and Beckwith-Wiedemann syndrome (Hatada et al. (1996) Nat Genet 14:171-173). The identification of inducers of alterations in imprinting can thus facilitate the discovery of potential new anti-cancer therapeutics. Similarly, in view of the presently disclosed differences in the imprinting profiles of VSELs in their quiescent state in tissues versus the imprinting profiles of VSEL derivatives present in VSEL-DSs, the presently disclosed subject matter also relates to assessing differences in imprinting profiles between quiescent cells (e.g., tissue VSELs) and pre-neoplastic and/or neoplastic derivatives thereof.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Employed in the EXAMPLES

Animals and preparation of BM cells for FACS. The studies disclosed herein were performed in accordance with the guidelines of the Animal Care and Use Committee of the University of Louisville School of Medicine (Louisville, Ky., United States of America) and with the Guide for the Care and Use of Laboratory Animals (United States Department of Health and Human Services Publication No. NIH 86-23). Murine mononuclear cells

(MNCs) were isolated from bone marrow (BM) of pathogen-free, 4-6 week-old female and male C57BL/6 mice. MNCs were also isolated from bone marrow of pathogen-free, 4-6 week old female and male heterozygous C57BU6-Tg(CAG-EGFP)1Osb/J transgenic mice (formerly C57BL/6-Tg(ACTB-EGFP)1Osb/J; Jackson Laboratory, Bar Harbor, Me., United States of America). These transgenic mice express an enhanced green fluorescent protein (eGFP) transgene under the transcriptional control of the chicken β-actin promoter and cytomegalovirus enhancer, which results in all tissues of the mice other than erythrocytes and hair expressing eGFP. BM cell suspensions isolated by flushing the marrow from bones were lysed in BD lysing buffer (BD Biosciences, San Jose, Calif., United States of America) for 15 minutes at room temperature (RT) and washed twice in phosphate buffered saline (PBS).

Isolation of VSELs from BM by FACS. VSELs (Lin^{neg}/Sca-1⁺/CD45^{neg}) and HSCs (Lin^{neg}/Sca-1⁺/CD45⁺) were isolated from BM cells isolated from 4-6 week-old mice by multiparameter, live cell sorting (FACSVANTAGE™ SE; Becton Dickinson, Mountain View, Calif., United States of America; or MOFLO™, Dako North America, Inc., Carpinteria, Calif., United States of America) as per Kucia at al., 2006b (Leukemia 20:857-869). Briefly, bone marrow mononuclear cells (BMMNCs) were resuspended at 10×10⁷ cells/ml in cell-sort medium (CSM) containing 1× Hank's Balanced Salt Solution without phenol red (GIBCO®, Grand Island, N.Y., United States of America), 2% heat-inactivated fetal calf serum (FCS; GIBCO®), 10 mM HEPES buffer (GIBCO®), and 30 unts/ml of Gentamicin (GIBCO®). The following monoclonal antibodies (mAbs) were employed for cell staining: biotinconjugated rat anti-mouse Ly-6A/E (Sca-1; clone E13-161.7); streptavidin-PE-Cy5 conjugate; anti-CD45-APC-Cy7 (clone 30-F11); anti-CD45R/B220-PE (clone RA3-6B2); anti-Gr-1-PE (clone RB6-8C5); anti-TCRab PE (clone H57-597); anti-TCRgz PE (clone GL3); anti-CD11b PE (clone M1/70); and anti-Ter-119 PE (clone TER-119). All mAbs were from BD Biosciences. mAbs were added at saturating concentrations and the cells were incubated for 30 minutes on ice, washed twice, then resuspended for sort in CSM at a concentration of 5×10⁶ cells/mi. The double-sorted populations of cells were employed.

Formation of VSEL-DSs and cell culture. The VSEL-DSs were cultured as previously described (Kucia at al. (2006a) Leukemia 20:857-869). Cells isolated from VSEL-DSs at days 5, 7, and 11 were employed. Murine ESC-D3 cells were purchased from the American Type Culture Collection (ATCC; Rockville, Md., United States of America) and grown in Dulbecco's modified Eagle's medium (DMEM; GIBCO®) containing 4 mM L-glutamine, 1.5 g/L sodium bicarbonate, 4.5 g/L glucose, 0.1 mM β-mercaptoethanol (Sigma-Aldrich Co., St Louis, Mo., United States of America), 15% heat-inactivated fetal bovine serum (FBS; GIBCO®), 100 IU/ml penicillin, 100 μg/ml streptomycin (INVITROGEN™, Carlsbad, Calif., United States of America), and 5 ng/ml of recombinant mouse Leukemia Inhibitory Factor (LIF; Chemicon-Millipore, Billerica, Mass., United States of America) without a feeder layer. Embryoid body (EB) formation was performed by the hanging drop method. The human hematopoietic cell line, THP-1, and murine BM stromal cells (STs) were maintained in RPMI 1640 (GIBCO®) and DMEM medium, respectively, supplemented with 10% FBS, 100 IU/ml penicillin, 100 μg/ml streptomycin, and 2 mM L-glutamine.

Carrier Chromatin-Immunoprecipitation (Carrier-ChIP). Carrier-ChIP analysis was performed as previously described (O'Neill et al. Nat Genet 2006; 38:835-841) with some modifications. Instead of Drosophila melanogaster SL2 cells, THP-1 cells were used as a source of carrier chromatin. The ChIP assay was performed using the MAGNA CHIP™ G kit (Upstate-Millipore, Billerica, Mass., United States of America) according to the manufacturer's instructions. In brief, 5×10⁶ THP-1 cells were resuspended in culture media and mixed with 2×10⁴ freshly isolated VSELs, HSCs, BMMNCs, ESC-D3s, or EB-derived cells. The cell mixtures were subsequently fixed with 1% formaldehyde in culture media for 10 minutes at RT with rotation. Excess formaldehyde was quenched by adding 10× glycine stock followed by incubation for 5 minutes at RT. The crosslinked chromatin in the cell mixtures was subsequently sheared by sonication (Model 150T, Fisher Scientific, Pittsburgh, Pa., United States of America) at 40% amplitude, four times 15 second pulse on with incubation at ice for 1 minute at intervals in 200 μl of Nuclear Lysis Buffer. After centrifugation at 10,000×g at 4° C. for 10 minutes, sheared chromatin was immunoprecipitated using Protein G magnetic beads conjugated to 3 μg of ChIP grade antibodies against H3Ac (Upstate-Millipore), H3K9me2 (Abcam, Cambridge, Mass., United States of America), or rabbit immunoglobulin (Ig) G control antibodies (Sigma-Aldrich). The bound and unbound sheared crosslinked chromatin was subsequently eluted according to the instructions provided with the MAGNA CHIP™ G kit.

PCR reactions were performed using AMPLITAQ® Gold Taq polymerase (Applied Biosystems, Foster City, Calif., United States of America), primers for murine sequence-specific Oct4, Nanog, or β-Actin promoter (see Table 1) as follows: a first incubation of 8 minutes at 95° C., a second incubation of 2 minutes at 95° C., 1 minute at the annealing temperature (AT), and 1 minute at 72° C. Subsequent to these pre-cycling incubations, the PCR reaction proceeded as follows: 30 seconds at 95° C.; 1 minute at the AT; and 1 minute at 72° C. After the number of cycles indicated in Table 1 hereinabove, the reactions were terminated with one cycle of 10 minutes at 72° C. The AT was 62° C. for the Oct4 reactions, 60° C. for the Nanog reactions, and 65° C. for the β-Actin reactions. Finally, PCR products were visualized by electrophoresis on 2% agarose gel.

To quantify the enrichment of each histone modification, RQ-PCR using the qChIP primer sets (see Table 1) was employed. The copy number of bound or unbound PCR products was calculated by the absolute quantification method. The enrichment of each histone modification was calculated as the ratio of amplicon amounts from bound (B) to unbound (UB) fractions and fold differences are shown as mean±S.D. from at least four independent experiments. All the clones obtained by employing these ChIP primers were subsequently sequenced to rule out the possibility of amplification of Oct4 pseudogenes or nonspecific sequences.

Bisulfite-sequencing and combined bisulfite-restriction analysis (COBRA). The DNA methylation statuses of the promoters of pluripotent regulators (Oct4, Nanog) and DMRs of imprinted-genes were investigated using bisulfite DNA modification followed by sequencing as well as by COBRA assay. In brief, genomic DNA were prepared from double-sorted VSELs, HSCs, STs, ESC-D3s, and cells derived from VSEL-DSs (2×10⁴) using the DNeasy Blood & Tissue Kit (Qiagen Inc., Valencia, Calif., United States of America). Next, 100 ng of gDNA were used in bisulfite modification, performed using the EpiTect Bisulfite Kit (Qiagen Inc.) according to the manufacturer's instructions. DMRs of imprinted genes were amplified by nested PCR using bisulfite treated gDNA and specific primers (see Table 1). Both first and second round PCR were performed at 2 cycles of 2 minutes at 95° C., 1 minute at 55° C., and 1 minute at 72° C., followed by 35 cycles of 30 seconds at 95° C., 1 minute at 55° C., 1 minute at 72° C., and 1 cycle of 10 minutes at 72° C. After agarose gel electrophoresis, amplicons were eluted using QIAquick Gel Extraction Kits (Qiagen Inc.). Eluted amplicons were subsequently ligated into pCR®2.1-TOPO® vector and transformed into TOP10 bacteria using a TOPO® TA Cloning Kit (INVITROGEN™). The plasmids were prepared using a QIAprep Spin Miniprep Kit (Qiagen Inc.) and sequenced with M13 forward and reverse primers. The methylation pattern in DMRs was analyzed using CpGviewer software (Carr et al. Nucl Acids Res 2007 May 11, 2007; 35(10):e79). The COBRA assay was performed by cutting amplicons of DMRs with Taql or BstUl restriction enzyme for 2 hours and subsequent agarose gel electrophoresis as previously described (Horii et al. Stem Cells 2008; 26:79-88). All experiments were conducted with three independent isolations of all the cell populations and two independent PCRs of each isolated cell population.

Reverse transcriptase-polymerase chain reaction (RT-PCR). Total RNA from various cells was isolated using the RNeasy Mini Kit (Qiagen Inc.) including DNase I treatment I (Qiagen Inc.). mRNA (10 ng) was reverse transcribed with TAQMAN® Reverse Transcription Reagents (Applied Biosystems) according to the manufacturer's instructions. The resulting cDNA fragments were amplified using AMPLITAQ® Gold with 1 cycle of 8 minutes at 95° C., 2 cycles of 2 minutes at 95° C., 1 minute at 60° C., and 1 minute at 72° C., followed by 35 cycles of 30 seconds at 95° C., 1 minute at 60° C., and 1 minute at 72° C., and 1 cycle of 10 minutes at 72° C. using the sequence specific primers set forth in Table 2 hereinabove. All primers were designed with PRIMER EXPRESS® software (Applied Biosystems), and at least one primer included an exon-intron boundary.

Real-time Quantitative PCR (RQ-PCR). Quantitative assessment of mRNA levels of target genes was performed by RQ-PCR using an ABI PRISM® 7500 Sequence Detection System (Applied Biosystems). cDNA templates from each cell were amplified using SYBR® Green PCR Master Mix (Applied Biosystems) and specific primers (see Table 2). All primers were designed with PRIMER EXPRESS® software (Applied Biosystems), and at least one primer included an exon-intron boundary. In case of Oct4 expression analysis, the primer set described by Lengner et al. ((2007) Cell Stem Cell 1:403-415) was employed (forward primer: 5′-ACATCGCCAATCAGCTTGG-3′ (SEQ ID NO: 97); reverse primer: 5′-AGAACCATACTCGAACCACATCC-3′ (SEQ ID NO 98)), and by sequencing the PCR products, the possibility of amplification of Oct4 pseudogenes or nonspecific sequences was excluded. The threshold cycle (Ct), defined as the cycle number at which the fluorescence of an amplified gene reached a fixed threshold, was subsequently determined and relative quantification of the expression level of target genes was performed with the 2^{-ΔΔCt} method, using the mRNA level of β2-microglobulin as an endogenous control and that of ST as a calibrator.

Immunocytochemistry. Immunocytochemistry with antibodies that were specific for Oct4, SSEA-1, p57^{KIP2} (polyclonal, Abcam Inc., Cambridge, Mass., United States of America), Dnmt1 (C-17, polyclonal, Santa Cruz, Santa Cruz, Calif., United States of America), and Dnmt3b (N-19, polyclonal, Santa Cruz) proteins was performed as previously described in Kucia et al. (Leukemia 2006a; 20:857-869).

Statistical Analyses. All the data in quantitative ChIP and gene expression analysis were analyzed using one factor ANOVA with Bonferroni's Multiple Comparison Test. The Instat1.14 program (GraphPad, La Jolla, Calif., United States of America) was employed, and statistical significance was defined as p<0.05 or p<0.01.

### EXAMPLE 1

### The Open Chromatin Structure of the Oct4 Promoter in VSELs

Whether Oct4⁺ cells are truly present in adult tissues is currently controversial (Liedtke et al. (2007) Cell Stem Cell 1:364-366; Lengner et al. (2007) Cell Stem Cell 1:403-415). These reports suggested that Oct4 expression in putative candidates of PSCs could merely be a result of detection of Oct4 pseudogenes by RT-PCR or unspecific staining. Therefore, Oct4 expression, if any, in candidate PSCs isolated from adult tissues was assayed.

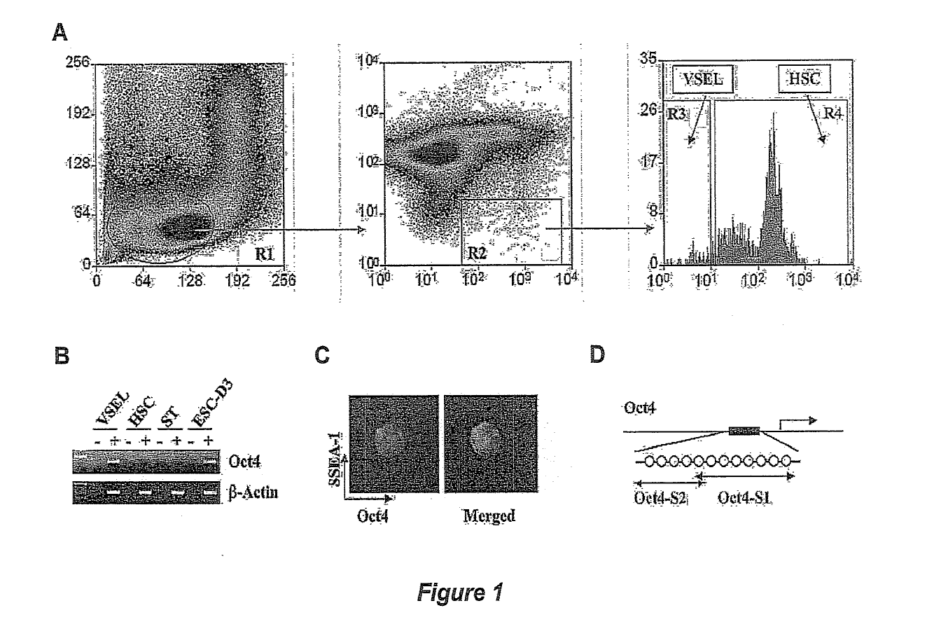

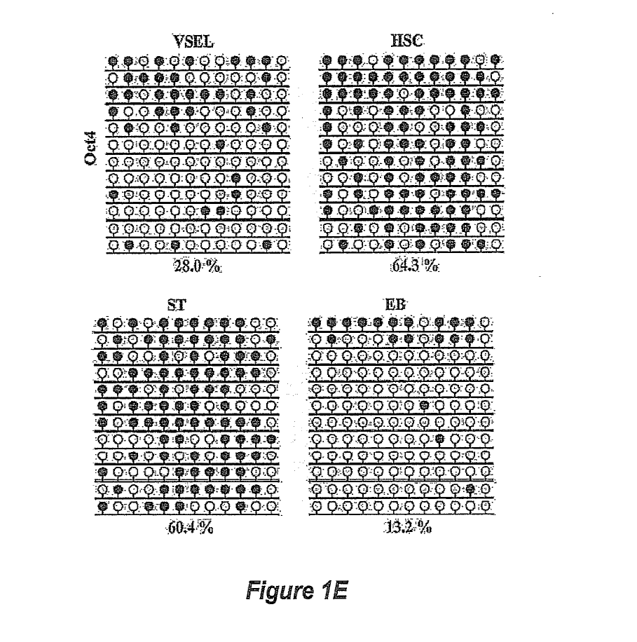

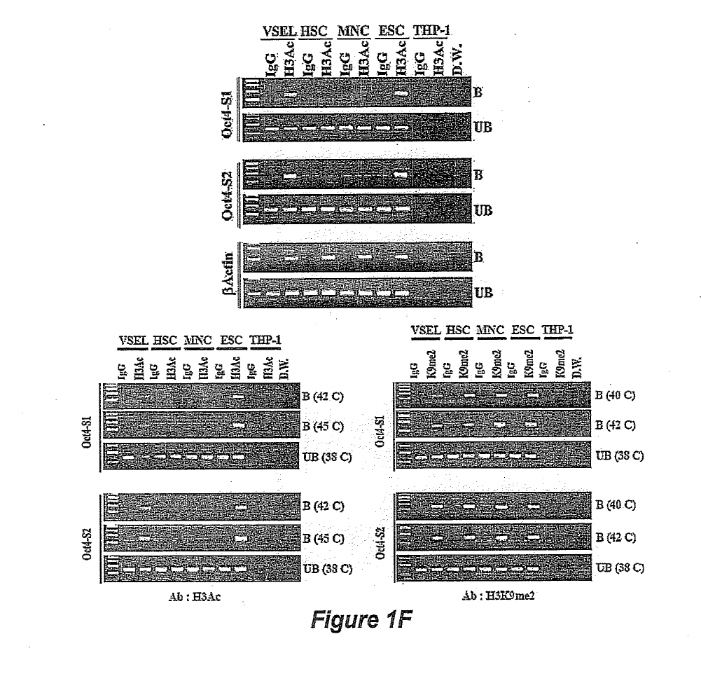

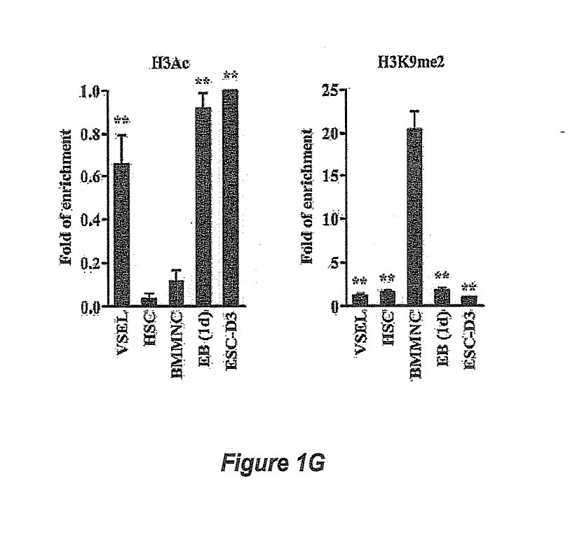

To determine whether VSELs expressed the Oct4 gene, the epigenetic status of the Oct4 promoter was examined in these cells. Lin^{neg}/Sca-1⁺/CD45^{neg} VSELs were double purified along with Lin^{neg}/Sca-1⁺/CD45⁺ hematopoietic stem cells (HSCs) by FACS (see FIG. 1A). First, that highly purified VSELs, similarly to ESC cell-line ESC-D3, expressed Oct4 both at the mRNA and protein levels was confirmed (see FIGS. 1B and 1C). Next, since expression of Oct4 is repressed in differentiated cells by a mechanism involving promoter methylation (Feldman et al. (2006) Nat Cell Biol 8:188-194), the DNA methylation status of the Oct4 promoter was examined (see FIG. 1D) by employing bisulfite-sequencing in murine VSELs, HSCs, BM-derived stromal cells (STs), and cells isolated from ESC-D3-derived 1-day embryoid bodies (EBs; see FIG. 1E). It was observed that the Oct4 promoter in VSELs, similar to that in EBs, was hypomethylated (28% and 13.2%, respectively). In contrast, the Oct4 promoter was hypermethylated in adult HSCs (63.4%) and STs (60.4%).

To provide additional direct evidence that the Oct4 promoter in VSELs was in an active/open state, carrier chromatin-immunoprecipitation (ChIP) assays were performed to evaluate the association of the Oct4 promoter with acetylated-histone3 (H3Ac) and dimethylated-lysine-9 of histone-3 (H3K9me2), the definitive molecular features for open- and closed-type chromatins, respectively (Margueron et al. (2005) Curr Opin Genet Dev 15:163-176). To overcome the challenges presented by low VSELs numbers, the Carrier-ChIP assay was performed using human hematopoietic cell-line THP-1 as carrier. As shown in FIG. 1F, Oct4 promoter chromatin was associated with H3Ac in both VSELs and ESC-D3 but not in primary HSCs, BM mononuclear cells (BMMNCs), or THP-1 cells, even in PCR reactions after employing high cycle numbers (FIG. 1F). Furthermore, RQ-PCR analysis of the ChIP products revealed that the Oct4 promoter in VSELs was highly enriched for H3Ac, which is similar to that seen in ESC-D3 and EB (1-day) cells, and its association with H3K9me2 was relatively very low (see FIG. 1G). Interestingly, in contrast to BM-derived MNCs, the Oct4 promoter in HSCs showed a weak association with both H3Ac and H3K9me2.

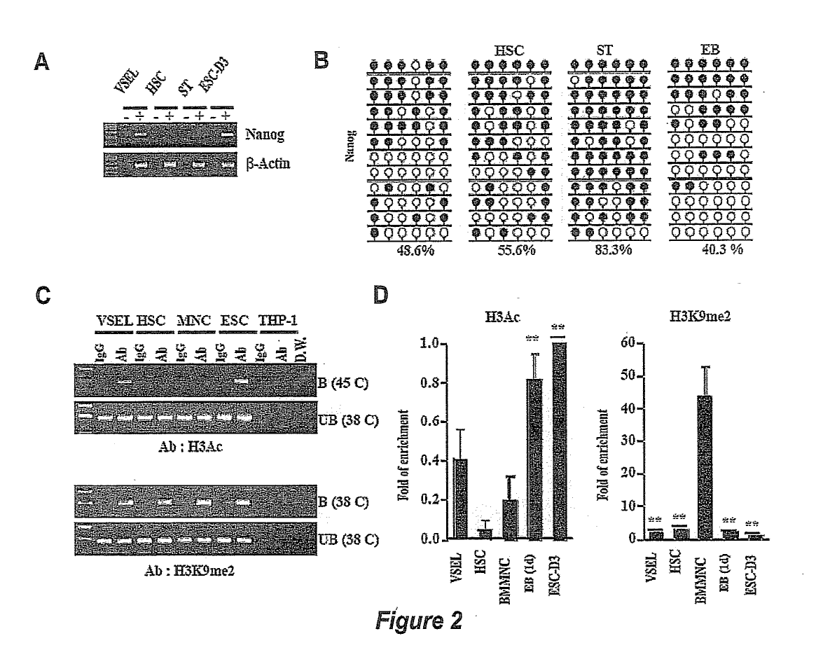

Since VSELs also express Nanog, the epigenetic status of the Nanog promoter was also determined in these cells. It was determined that the Nanog promoter was methylated (˜50%); however, quantitative ChIP data confirmed that the H3Ac/H3K9me2 ratio supported the active status of the Nanog promoter in VSELs (FIG. 2). Thus, VSELs appeared to express both Oct4 and Nanog.

### Example 2

### Unique Genomic Imprinting Patterns Result in a Quiescent Transcriptome in VSELs

Unlike ES cells, highly purified BM-derived VSELs do not proliferate in vitro if cultured alone. Based on the expression of PSCs markers and primitive morphology, it was possible that the quiescence of VSELs could be controlled by erasure/modification of methylation on some developmentally important imprinted genes in a manner similar to that observed in epiblast-derived PGCs (Ratajczak et al. (2007) Leukemia 21:860-867). To test whether VSELs undergo epigenetic reprogramming and/or modification of genomic imprinting, the DNA methylation status on DMRs of paternally-methylated imprinted genes (Igf2-H19, Rasgrf1, and Meg3) was tested (see FIG. 3A). The rationale was that paternally imprinted genes are rare (Kobayashi et al. Cytogenet Genome Res 113:130-137) and, additionally, the proper mono-allelic imprint of the Igf2-H19 genes plays a role in obtaining viable parthenogenetic mice derived from a reconstructed oocyte containing two haploid sets of maternal genomes (Kono et al. (2004) Nature 428:860-864).

VSELs showed significant hypomethylation (˜10%) of the DMR for Igf2-H19 locus (see FIG. 3B). In contrast, this region was normally methylated (˜50%) in HSCs and STs, and even slightly hypermethylated in ESC-D3 (see FIG. 3B). These bisulfite-sequencing results were subsequently confirmed by combined bisulfite-restriction analysis (COBRA; see FIG. 3E).

Next, the methylation status of DMRs for Rasgrf1 and Meg3 were assayed, and it was determined that VSELs, in contrast to other cells, erased imprinting on the DMR for Rasgrf1 (see FIG. 3C). However, the DMR for Meg3 was properly methylated (see FIG. 3D), indicating that VSELs erased the genomic imprinting on DMRs for paternally imprinted Igf2-H19 and Rasgrf1 loci similarly to that observed in PGCs (Hajkova et al. (2002) Mech Dev 117:15-23), but not at the DMR for Meg3.

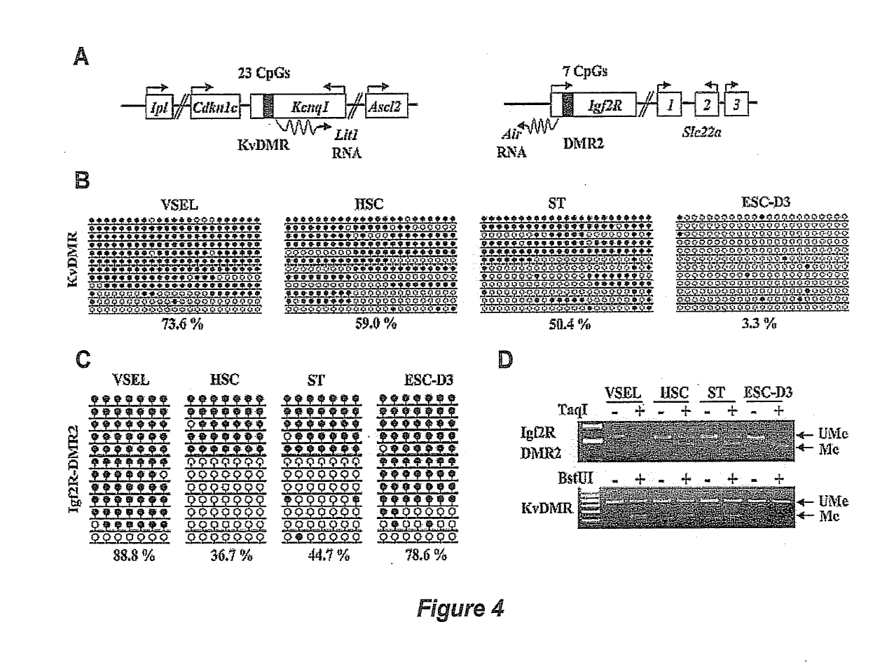

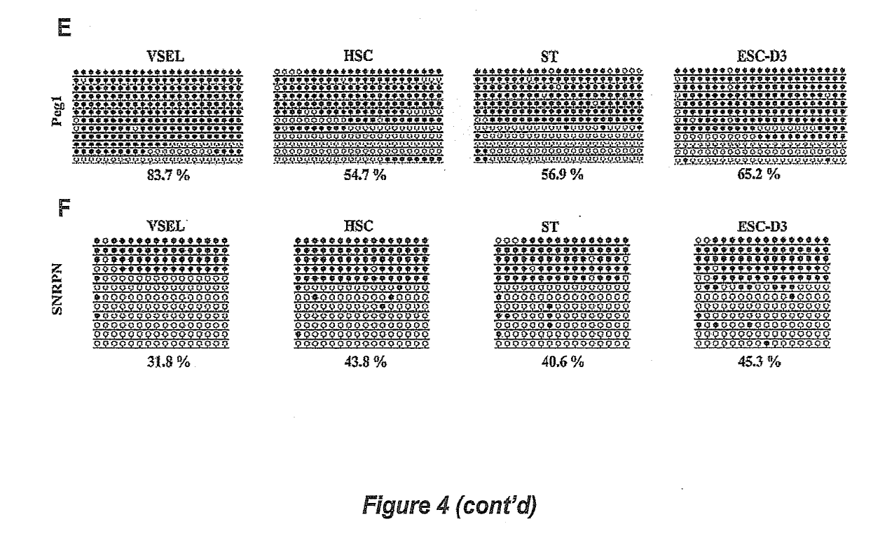

Next, DMRs for selected maternally methylated loci (Kcnq1, Igf2R) that have been implicated in the regulation of embryo growth were studied (see FIG. 4A). DMRs for both maternally imprinted loci, Kcnq1 (see FIG. 3B) and Igf2R (see FIG. 4C) were both hypermethylated in VSELs. At the same time, all these regions were normally methylated (˜50%) in adult HSCs and STs. Highly proliferative ESC-D3 cells showed opposite methylation patterns in DMRs for the Kcnq1 (see FIG. 4B), Igf2-H19 (see FIG. 3B), and Rasgrf1 (see FIG. 3C) loci compared to VSELs. These bisulfite-sequencing results were subsequently confirmed by COBRA assay (see FIG. 4D). When other maternally methylated genes (Peg1, SNRPN) were investigated, it as found that the DMR for Peg1 was hypermethylated (see FIG. 4E); however, the DMR for SNRPN was slightly hypomethylated (see FIG. 4F) in VSELs as compared to other cells.

Thus, the DMR methylation results disclosed herein revealed a unique genomic imprinting pattern in VSELs, showing a tendency to erase paternally methylated DMRs but hypermethylation of maternally methylated DMRs. It is accepted that while paternally expressed imprinted genes (e.g., Igf2, Rasgrf1) enhance the growth of embryos, maternally expressed imprinted genes (e.g., H19, p57^{KIP2}, Igf2R) inhibit cell proliferation (Reik & Walter (2001) Nat Rev Genet 2:21-32). Therefore, the differences observed on VSELs demonstrate growth-repressive imprints in these cells.

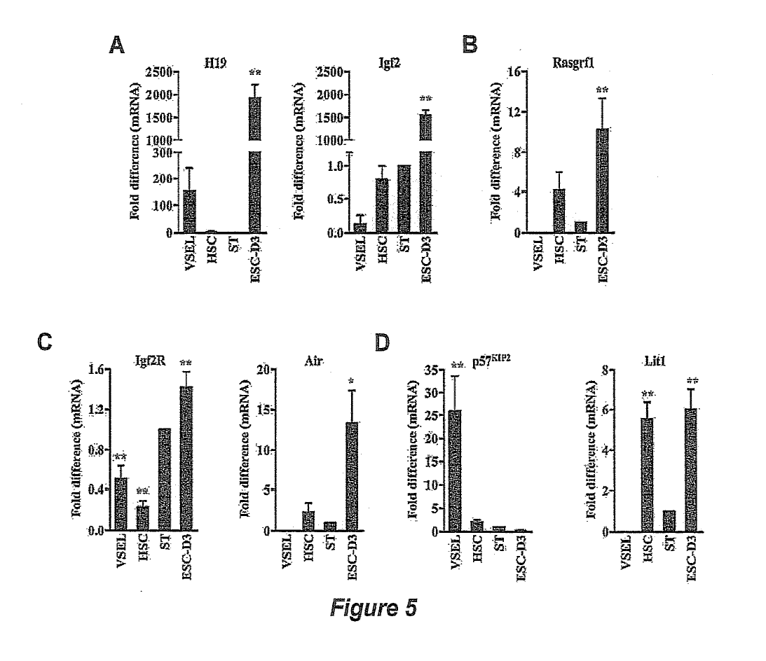

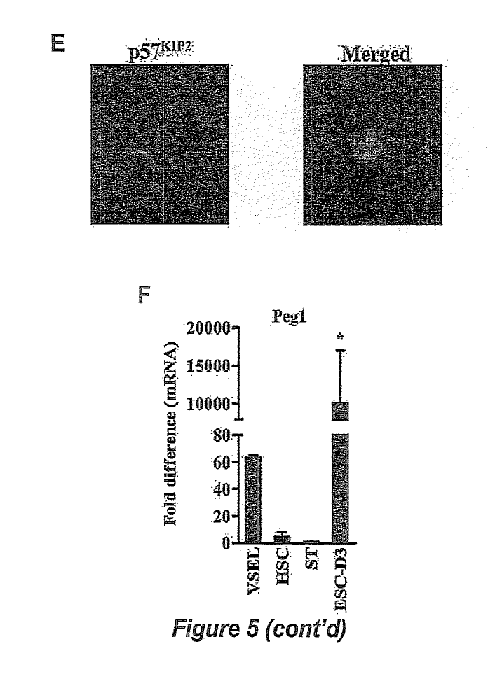

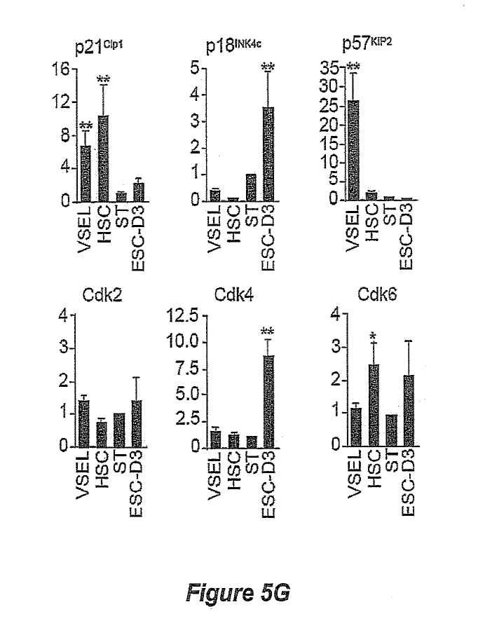

To confirm the DMR methylation results, RQ-PCR analysis of the expression of imprinted genes was performed. VSELs were found to downregulate mRNA for Igf2 (see FIG. 5A) and Rasgrf1 (see FIG. 5B) while simultaneously highly upregulated H19 (see FIG. 5A). H19 and Igf2 were found to be highly expressed in ESC-D3 cells (see FIG. 5A), and the ratio of H19/Igf2 mRNA for VSELs and ESC-D3 was about 400:1 vs. about 1:1, respectively.

In contrast to CCCTC-binding factor-(CTCF) regulated genes (Igf2-H19, Rasgrf1), a different mechanism regulates expression of Igf2R and Kcnq1 (Delaval & Feil (2004) Curr Opin Genet Dev 14:188195). DMRs for these loci are located in promoters of antisense transcripts (Air and Lit1, respectively) that coordinately repress expression of clustered imprinted genes (see FIG. 4A). As demonstrated for hypermethylation of DMRs for Igf2R and Kcnq1 (see FIG. 4), VSELs downregulated expression of Air and Lit1 (see FIGS. 5C and 5D). As a result, VSELs highly expressed Ig2R (see FIG. 5C) and, more importantly, highly upregulated p57^{KIP2}, a known negative regulator of the cell cycle (see FIGS. 5D and 5E).

In addition to p57^{KIP2},l other cyclin-dependent kinases (Cdks) or inhibitors (CDKIs) were also assayed. High expression of p21^{Cip1} was observed, but no significant differences in the expression level of Cdks2, 4, and 6 were seen, suggesting that CDKIs could play more important roles in VSELs quiescence than Cdks (see FIG. 5G).

Some of the DMRs of imprinted genes are located directly in their promoters (e.g., Peg1). Although the Peg1 DMR was hypermethylated in VSELs, these cells highly expressed mRNA for this gene (see FIG. 5F), similar to that observed for ESC-D3 cells. A high level of Peg1 expression was also observed in VSELs, which is similar to that observed in embryonic cells (Lefebvre et al. (1998) Nat Genet 20:163-169).

Based on the data disclosed herein, it appeared that epigenetic reprogramming of genomic imprinting in VSELs resulted in a quiescent transcriptome in these cells by upregulation of growth-repressive genes (H19, p57^{KIP2}, Igf2R) and downregulation of growth-promoting genes (Igf2, Rasgrf1). Therefore, these changes in methylation patterns suggest a mechanism involved in regulating the pluripotency of early developmental stem cells deposited in adult tissues.

### EXAMPLE 3

### VSELs Highly Express Dnmts

Since the methylation status of imprinted genes and their expression is regulated by Dnmts (Dnmt1, Dnmt3b, Dnmt3a, Dnmt3L), expression of these genes in VSELs was assayed. Dnmt1 is believed to play a role in the maintenance of DNA methylation and Dnmt3a and 3b are accountable for de novo DNA methylation (Chen et al. (2003) Mol Cell Biol 23:5594-5605). Furthermore, Dnmt3L, a gene that shares homology with the Dnmt3 family methyltransferases despite its lack of enzymatic activity, also plays a role in DNA methylation of imprinted genes (Bourc'his et al. (2001) Science 294:2536-2539).

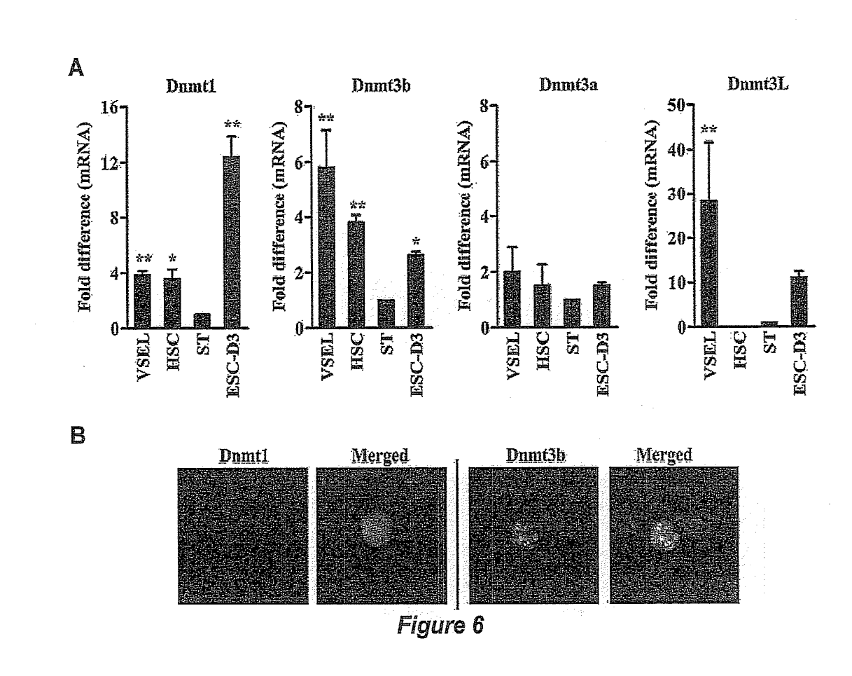

The present data revealed that VSELs highly expressed all Dnmts, similar to ESCs, and in particular, were highly enriched for mRNA for Dnmt3L (see FIG. 6A). Intranuclear expression of Dnmt1 and Dnmt3b in VSELs was observed by immunostaining (see FIG. 6B). Because the expression of de novo Dnmts and Dnmt3L is low in differentiated somatic cells, high expression of these Dnmts in VSELs suggested their high epigenetic plasticity, similar to that observed for ESCs.

### EXAMPLE 4

### The Reprogramming of Genomic Imprinting During VSEL-DSs Formation

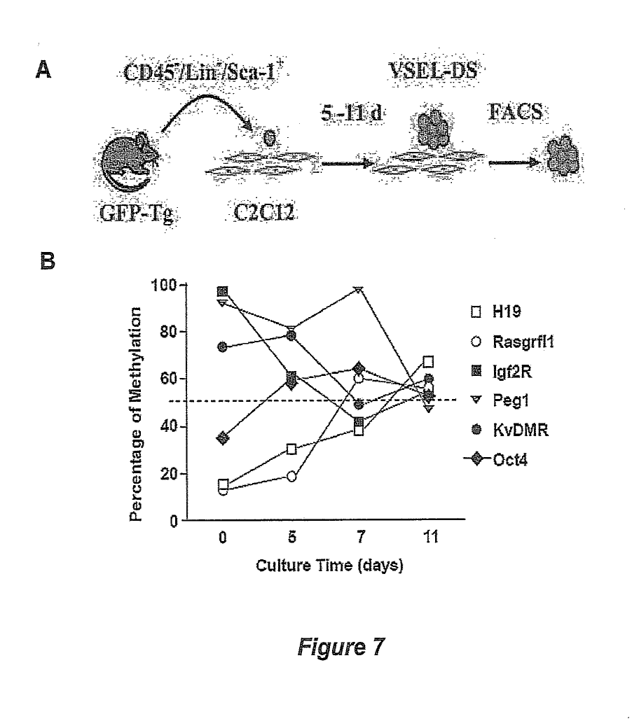

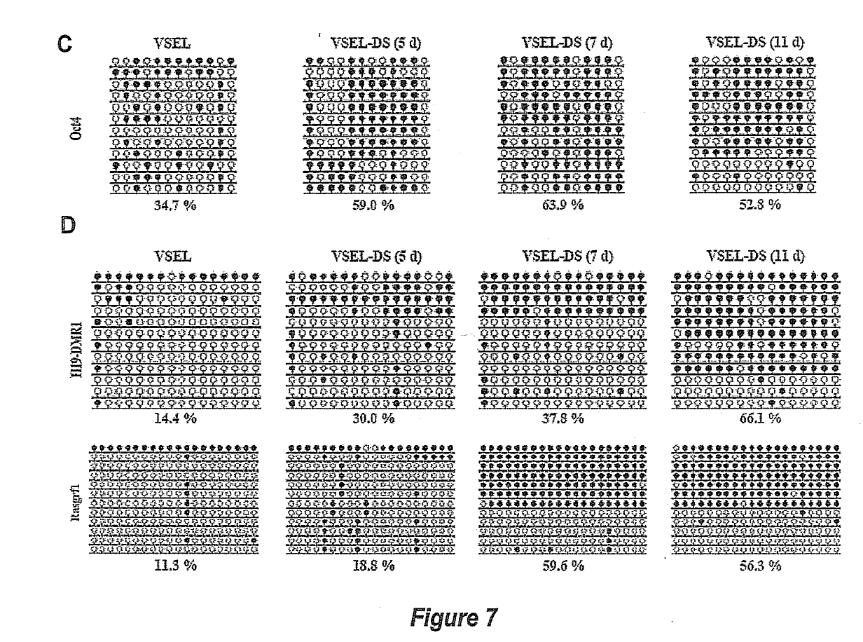

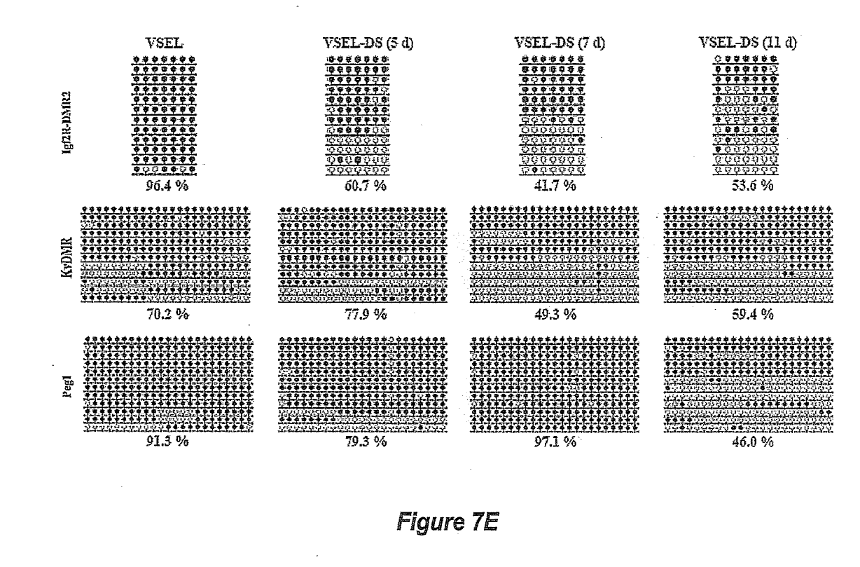

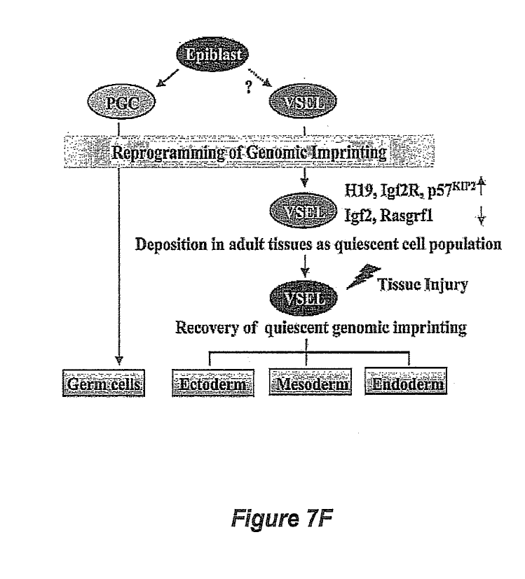

Although purified VSELs remain quiescent if cultured alone in vitro, they can generate VSEL-DSs in co-cultures with C2C12 (Kucia et al. (2006a) Leukemia 20:857-869). To test whether the quiescent status of VSELs can be modulated by epigenetic reprogramming in VSELs after cell-to-cell contact with the C2C12 supportive cell line, the DNA methylation of the Oct4 promoter and selected imprinted genes was assayed in cells isolated from VSEL-DSs at days 5, 7, and 11 (see FIG. 7A). Both gradual hypermethylation of the Oct4 promoter and occurrence of somatic methylation pattern on DMRs were observed for Igf2-H19, Rasgrf1, Igf2R, Kcnq1, and Peg1 (see FIGS. 7B-7E).

The results summarized in FIG. 7B suggest that growth-repressive genomic imprinting in VSELs is gradually restored in stem cells that form VSEL-DS, which suggested that VSELs might show dynamic epigenetic plasticity potential similar to that seen in ESCs. However, restoration of genomic imprints in cells isolated from VSEL-DSs was paralleled by hypermethylation of the Oct4 promoter (see FIG. 7B).

Therefore, the results presented herein demonstrated that the DNA methylation of Oct4 promoter and DMRs in certain imprinted genes together played a role in the pluripotent and quiescent statuses of VSELs (see FIG. 7F).

### Discussion of the EXAMPLES

The present study for the first time provides molecular evidence at chromatin level that Oct4 gene is actively transcribed in VSELs isolated from adult BM. In addition, the methylation studies of the Oct4 promoter and imprinted genes disclosed herein reveal novel mechanisms that might prevent “unleashed” proliferation of developmentally early stem cells deposited in adult tissues. VSELs show some similarities in methylation pattern to PGCs, which suggests their close relationship to epiblast/germ line cells (FIG. 7F).

Some recent reports cast some doubts if Oct4 could be truly expressed in cells isolated from adult tissues and prompt us to reappraise expression of Oct4 in VSELs. To rule out that Oct4 expression could result from misinterpretation of RT-PCR and immunostaining results, Oct4-specific primers that do not amplify pseudogenes were employed; contaminating genomic DNA was removed during RNA isolation with Deoxyribonuclease I (DNase I) treatment; the PCR products were confirmed by DNA sequencing; and the intranuclear localization of Oct4 protein was shown. Additionally, epigenetic analysis (DNA methylation and histone modifications) of the Oct4 promoter was also performed (see FIG. 1). The data presented herein provide strong molecular evidence at the chromatin level that the Oct4 gene is truly transcribed in VSELs isolated from adult BM. Similarly, using similar approaches, evidence for an open status of the Nanog promoter is also presented herein.

Furthermore, in the present study, the hypothesis that VSELs might show some unique genomic imprinting patterns that regulate their quiescent status was confirmed. The role of status of genomic imprinting in regulation of pluripotentiality was described for another epiblast-derived stem cell population, PGCs. These germline committed stem cells gradually reprogram/erase their genomic imprinting during migration to genital ridges between developmental days 8.5-12.5 post coitus (dpc; Hajkova et al. (2002) Mech Dev 117:15-23). Erasure of genomic imprint in PGCs results in pluripotent cells that i) are quiescent; ii) do not complete blastocyst development; and iii) include nuclei that, in contrast to any other somatic cell nuclei, are ineffective as DNA donors for nuclear transfer (Yamazaki et al. (2003) Proc Natl Acad Sci USA 100:12207-12212). It was further observed that in VSELs, paternally methylated DMRs (Igf-2-H19 and Rasgrf1) were hypomethylated as are PGCs, in contrast to maternally methylated ones (Kcnq1, Igf2R, and Peg1) that remained hypermethylated in VSELs (see FIGS. 3 and 4).

The methylation status of DMRs is regulated by Dnmts. While PGCs express Dnmt1, both Dnmt3a and Dnmt3L are not expressed in these cells (Hajkova et al. (2002) Mech Dev 117:15-23; Durcova-Hills et al. (2008) PLoS ONE 3:e3531). Furthermore, in contrast to PGCs, VSELs highly expressed all types of Dnmts (see FIG. 6), particularly Dnmt3L, which plays a role in establishing maternal methylated imprints (Bourc'his et al. (2001) Science 294:2536-2539).

Thus, the high expression of DNA methylation machinery in VSELs could explain the hypermethylation statuses of maternally imprinted DMRs. However, despite of potential high capacity of DNA methylation in these cells, it was observed that paternally-methylated DMRs for the Igf2-H19 and Rasgrf1 loci were erased. To explain this striking discrepancy, it was determined that VSELs highly expressed the chromatin insulator CTCF gene that has been implicated in preventing methylation of paternally imprinted DMRs.

Therefore, high CTCF expression could protect against the methylation of paternally-methylated DMRs in these cells. This notion is further supported by the additional observation that VSELs showed a slight hypomethylation in DMR for SNRPN, which is also regulated by CTCF (Pant et al. (2003) Genes Dev 17:586-590). Based on these observations, it appears that a balance between Dnmts and CTCF expression can influence a final outcome of DMR methylation patterns in VSELs.

Imprinted genes are known to play roles in fetal growth, development, and tumorigenesis. As a likely result of unique reprogramming of genomic imprinting, VSELs show upregulation of growth-repressive imprinted genes (H19, p57^{KIP2}, Igf2R) and downregulation of growth-promoting genes (Igf2, Rasgrf1; see FIG. 5). Since Igf2 has been described as an important autocrine growth-factor that promotes expansion of several cell types (see e.g., Eggenschwiler et al. (1997) Genes Dev 1997; 11:3128-3142) and, in contrast, H19 regulatory mRNA has been found to inhibit cell proliferation (Hao et al. (1993) Nature 365:764-767), the changes in expression of both these genes were likely responsible for a quiescent status of VSELs.

Another gene that is downregulated by changes in DMRs methylation is Rasgrf1, which encodes a protein involved in Igf1R signal transduction (Font de Mora et al. (2003) EMBO J 22:3039-3049). Thus, the data disclosed herein support the notion that VSELs showed some changes in the expression of genes that were related to Igf signaling machinery.

Furthermore, it was observed that VSELs highly expressed transcript as result of hypermethylation of DMR in Kcnq1 locus. These results suggest that p57^{KIP2} could also play a role in maintaining VSELs quiescence. The data disclosed herein also demonstrated that all the observed changes in genomic imprinting that affected the pluripotent and quiescent statuses of VSELs could become reverted when these cells were expanded/differentiated into VSEL-DSs.

Thus, provided herein is molecular evidence that some rare Oct4⁺ VSELs are present in adult tissues. These cells could serve as a backup for tissue committed monopotent SCs, and their proliferative potential is tightly regulated by both Oct4 expression and the methylation status of some imprinted genes that are directly involved in regulation of cell proliferation (e.g., Igf2, H19, Igf2R, p57^{KIP2}, Rasgrf1).

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Anderson et al. (1999) Neoplasia 1: 340-348.
Ausubel et al. (2002) Short Protocols in Molecular Biology, Fifth ed. Wiley, New York, N.Y., United States of America.
Ausubel et al. (2003) Current Protocols in Molecular Biology, John Wylie & Sons, Inc., New York, N.Y., United States of America.
Blelloch et al. Stem Cells 2006; 24:2007-2013.
Bourc'his et al. (2001) Science 294:2536-2539.
Caplan et al. (2001) 7 Trends Mol Med 259-264.
Carr et al. (2007) Nucl Acids Res 35(10):e79.
Casola et al. (1997) Oncogene 14:1503-1510.
Castro et al. (2002) 297 Science 1299.
Chen et al. (2003) Mol Cell Biol 23:5594-5605.
Corti et al. (2002) 277 Exp Cell Res 74-85.
De Souza et al. (1997) FASEB J 11:60-67.
Delaval & Feil (2004) Curr Opin Genet Dev 14:188-195.
Demetrick et al. (1995) Cytogenet. Cell Genet 69:190-192.
D'Ippolito et al. (2004) J Cell Sci 117:2971-2981.
Durcova-Hills et al. (2008) PLoS ONE 3:e3531.
Eggenschwiler et al. (1997) Genes Dev 1997; 11:3128-3142.
Evans & Kaufman (1981) Nature 292:154-156.
Feldman et al. (2006) Nat Cell Biol 8:188-194.
Font de Mora et al. (2003) EMBO J 22:3039-3049.
Fournier et al. (2002) EMBO J 21:6560-6570.
Geiger et al. 100 Blood 721-723.
GENBANK® Accession Nos. AAB25223; AAR16420; NC_{—}000001; NC_{—}000006; NC_{—}000007; NC_{—}000011; NC_{—}000014; NC_{—}000015; NM_{—}000076; NM_{—}000218; NM_{—}000389; NM_{—}000612; NM_{—}000876; NM_{—}001262; NM_{—}002402; NM_{—}002891; NM_{—}003097; NP_{—}000209; NP_{—}000380; NP_{—}000603; NP_{—}000867; NP_{—}001009318; NP_{—}001253; NP_{—}002393; NP_{—}002393; NP_{—}002829; NP_{—}002882; NP_{—}003088; NP_{—}034868; NP_{—}035340; NP_{—}598415; NP_{—}612516; NP_{—}776434; NP_{—}999251; NR_{—}002196; NR_{—}002766; XP_{—}002829; XP_{—}223083; XP_{—}599431.
Hajkova et al. (2002) Mech Dev 117:15-23.
Hao et al. (1993) Nature 365:764-767.
Hao et al. (2003) 12 J Hematother Stem Cell Res 23-32.
Harlow & Lane (1988) Antibodies: A Laboratory Manual, Cold Spring Harbor Laboratory, Cold Spring Harbor, N.Y.
Harlow & Lane (1999) Using Antibodies: A Laboratory Manual, Cold Spring Harbor Laboratory, Cold Spring Harbor, N.Y.
Hatada et al. (1996) Nat Genet 14:171-173.
Hattori et al. (2004) J Biol Chem 279:17063-17069.
Haynesworth et al. (1992) 13 Bone 81-88.
Holden & Vogel (2002) 296 Science 2126-2129.
Holm et al. (2005) Cancer Cell 8:275-285.
Horii et al. (2008) Stem Cells 26:79-88.
Hu et al. (1998) Mol Endocrinol 12:220-232.
Janus et al. (2003) 111 J Clin Invest 843-850.
Jiang et al. (2002) Nature 418:41-49.
Kato et al. (2007) Hum Mol Genet 16:2272-2280.
Kawada & Ogawa (2001) 98 Blood 2008-2013.
Kobatake et al. (2004) Oncol Rep 12:1087-1092.
Kobayashi et al. (2006) Cytogenet Genome Res 113:130-137.
Kono et al. (2004) Nature 428:860-864.
Korbling et al. (2002) 346 N Engl J Med 738-746.
Kucia et al. (2005) Leukemia 20:18-28.
Kucia et al. (2006a) Leukemia 20:857-869.
Kucia et al. (2006b) Leukemia 21:297-303.
Labarge & Blau (2002) 111 Cell 589-601.
Lee & Stoffel (2003) 111 J Clin Invest 799-801.
Lee et al. (1995) Genes Dev 9:639-649.
Lefebvre et al. (1998) Nat Genet 20:163-169.
Lemischka (2002) 30 Exp Hematol 848-852.
Lengner et al. (2007) Cell Stem Cell 1:403-415.
Liedtke et al. (2007) Cell Stem Cell 1:364-366.
Lopes et al. (2003) Hum Mol Genet 12:295-305.
Lucifero et al. (2002) Genomics 79:530-538.
Mackay et al. (1998) 4 Tissue Eng 415-428
Mager et al. (2003) Nat Genet 33:502-507.
Makino et al. (1999) 103 J Clin Invest 697-705.
Margueron et al. (2005) Curr Opin Genet Dev 15:163-176.
Matsui et al. Cell 70:841-847.
McKinney-Freeman et al. (2002) 99 Proc Natl Acad Sci USA 1341-1346.
Miyoshi et al. (2000) Genes Cells 5:211-220.
Morison et al. (2005) Trends Genet 21:457-465.
Murphy & Jirtle (2003) Bioessays 25:577-588.
O'Neill et al. (2006) Nat Genet 38:835-841.
Orlic et al. (2003) 7 Pediatr Transplant 86-88.
Paczkowska et al. (2009) Stroke 40:1237-1244.
Pannetier& Feil (2007) Trends Biotechnol 25:556-562.
Pant et al. (2003) Genes Dev 17:586-590.
PCT International Patent Application Publication Nos. WO 2007/067280 and 2009/059032.
Petersen et al. (1999) 284 Science 1168-1170.
Pittenger et al. (2000) 251 Curr Top Microbiol Immunol 3-11.
Ratajczak et al. (2007) Leukemia 21:860-867.
Reed & Leff (1994) Nat Genet 6:163-167.
Reik & Walter (2001) Nat Rev Genet 2:21-32.
Reule et al. (1998) Dev Genes Evol 208:161-163.
Reyes & Verfaillie (2001) 938 Ann NY Acad Sci 231-235.
Reyes et al. (2001) 98 Blood 2615-2625.
Sadri et al. (1996) Nucleic Acids Res (1996) 24:4987-4989
Sambrook & Russell (2001) Molecular Cloning: A Laboratory Manual, 3rd edition, Cold Spring Harbor Laboratory Press, Cold Spring Harbor, N.Y.
Sanchez-Ramos (2002) 69 Neurosci Res 880-893.
Schwartz et al. (2000) 109 J Clin Invest 1291-1302.
Serrano et al. (1993) Nature 366:704-707.
Stamm et al. (2003) 361 Lancet 45-46.
Takahashi & Yamanaka (2006) Cell 126:663-676.
Tesar et al. (2007) Nature 448:196-199.
Thakur et al. (2004) Mol Cell Biol 24:7855-7862.
Thomassin et al. (1999) Methods 19:465-475.
Tijssen (ed.) (1993) Laboratory Techniques in Biochemistry and Molecular Biology: Hybridization With Nucleic Acid Probes, Part I Theory and Nucleic Acid Preparation, Elsevier Press, New York, N.Y., United States of America.
Trouillard et al. (2004) Cancer Genet Cytogenet 151:182-183.
U.S. Pat. No. 7,186,512.
Wagers et al. (2002) 297 Science 2256-2259.
Warnecke et al. (1990) Genomics 51:182-190.
Weber et al. (2001) Mech Devel 101:133-141.
Wojakowski et al. (2009) J Am Coll Cardio 53:1-9.
Xiong & Laird (1997) Nucleic Acids Res 25:2532-2534.
Yamazaki et al. (2003) Proc Natl Acad Sci USA 100:12207-12212.
Yatsuki et al. (2002) Genome Research 12:1860-1870.
Yoo et al. (1998) 80 J Bone Joint Surg Am 1745-1757.
Yoon et al. (2005) Moll Cell Biol 25:11184-11190.
Young et al. (1998) 16 J Orthop Res 406-413.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for determining a degree of pluripotency in a first putative stem cell relative to a second putative stem cell, the method comprising comparing imprinting statuses of one or more loci selected from the group consisting of Igf2-H19, Rasgrf1,Igf2R, Kcnq1, and Peg1/Mest between the first putative stem cell and the second putative stem cell, wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus are indicative of a more pluripotent state.

2. The method of claim 1, wherein the first and second putative stem cells are selected from the group consisting of very small embryonic like stem cells (VSELs), hematopoietic stem cells (HSCs), and mesenchymal stem cells (MSCs).

3. A method for distinguishing a very small embryonic like stem cell (VSEL) from a hematopoietic stem cell (HSC) or an mesenchymal stem cell (MSC), the method comprising comparing an imprinting status of one or more loci of the VSEL selected from the group consisting of Igf2-H19, Rasgrf1, Igf2R, Kcnq1, and Peg1/Mest to the same one or more loci in an HSC or an MSC, wherein hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus relative to levels of methylation at these loci in an HSC or an MSC are indicative of VSELs.

4. A method for isolating a very small embryonic like stem cell (VSEL) from a source expected to comprise VSELs, the method comprising:
(a) isolating a plurality of CD45^{neg}/lin^{neg} cells that are Sca-1⁺ or CD34⁺ from the source; and
(b) isolating a subset of cells from the plurality of CD45^{neg}/lin^{neg} cells, that are Sca-1⁺ or CD34⁺, wherein the subset of cells are characterized by one or more of hypomethylation at the Igf2-H19 locus, hypomethylation at the Rasgrf1 locus, hypermethylation at the Igf2R locus, hypermethylation at the Kcnq1 locus, and hypermethylation at the Peg1/Mest locus as compared to the fraction of cells remaining in the plurality of CD45^{neg}/lin^{neg} cells that are Sca-1⁺ or CD34⁺.

5. The method of claim 4, further comprising fractionating the cells to identify cells that are Oct-4⁺, CXCR4⁺, and/or SSEA-1⁺.

6. The method of any one of claims 1, 3, and 4, wherein the hypomethylation at the Rasgrf1 locus comprises hypomethylation at a differentially methylated region (DMR) of the Rasgrf1 promoter, the hypermethylation at the Igf2R locus comprises hypomethylation at a DMR2 region of the IgfR2 promoter, the hypermethylation at the Kcnq1 locus comprises hypermethylation of a KvDMR region of the Kcnq1 promoter, and/or the hypermethylation at the Peg1/Mest locus comprises hypermethylation of a DMR region of the Peg1/Mest promoter.

7. A kit comprising a plurality of oligonucleotide primers, wherein the oligonucleotide primers specifically bind to a subsequence of a differentially methylated region (DMR) in a nucleic acid or bind to a nucleotide sequence that flanks a DMR in a nucleic acid, wherein the oligonucleotide primers can be used to assay the methylation status of at least one methylated nucleotide present within the DMR.

8. The kit of claim 7, wherein the DMR is a human DMR.

9. The kit of claim 7, wherein the DMR is present in a locus selected from the group consisting of an Igf2-H19 locus, a Rasgrf1 locus, an Igf2R locus, a Kcnq1 locus, and a Peg1/Mest locus.

10. The kit of claim 7, wherein the plurality of oligonucleotide primers are designed to assay the DMR using a technique selected from the group consisting of bisulfite sequencing, carrier chromatin-immunoprecipitation (ChIP), and quantitative ChIP (qChIP).

11. The kit of claim 10, wherein at least one of the plurality of oligonucleotides primers comprises a nucleotide sequence of any of SEQ ID NOs: 1-96.

12. A method for assessing the purity of a very small embryonic like stem cell (VSEL) preparation, the method comprising:
(a) providing a first preparation suspected of comprising VSELs; and
(b) comparing an imprinting profile of cells of the first preparation with respect to one or more loci selected from the group consisting of Igf2-H19, Rasgrf1, Igf2R, Kcnq1, and Peg1/Mest to an imprinting profile of a second preparation of VSELs with respect to the same one or more loci,
wherein relative to the second preparation, hypermethylation at the Igf2-H19 locus, hypermethylation at the Rasgrf1 locus, hypomethylation at the Igf2R locus, hypomethylation at the Kcnq1 locus, and hypomethylation at the Peg1/Mest locus relative to levels of methylation at these loci in the second preparation is indicative of the first preparation being less pure with respect to VSELs than the second preparation.

13. The method of claim 12, further comprising isolating the first preparation from a source that comprises VSELs and at least one other stem cell type selected from the group consisting of hematopoietic stem cells (HSCs) and mesenchymal stem cells (MSCs).
